(19) United States
(12) Patent Application Publication
(10) Pub. No.: US 2024/0168776 A1
(43) Pub. Date: May 23, 2024

(54) DYNAMIC INTERFACES BASED ON MACHINE LEARNING AND USER STATE

(71) Applicant: MatrixCare, Inc., Bloomington, MN (US)

(72) Inventor: Kedar Mangesh KADAM, Nova Scotia (CA)

(21) Appl. No.: 18/497,695

(22) Filed: Oct. 30, 2023

Related U.S. Application Data
(60) Provisional application No. 63/383,432, filed on Nov. 11, 2022.

(51) Int. Cl. G06F 9/451; G16H 40/20; G16H 50/30

(52) U.S. Cl. CPC G06F 9/451; G16H 40/20; G16H 50/30

(57) ABSTRACT

Techniques for improved dynamic interfaces are provided. User interaction, from a user, is received via a graphical user interface (GUI) of a computing device. In response to receiving the user interaction, a set of user data associated with the user is collected, and a first stress score is generated by processing the set of user data using a stress model. In response to determining that the first stress score satisfies one or more defined criteria, a first prompt is generated for the user, where the first prompt requests additional user interaction, as compared to a default prompt, and the first prompt is output via the GUI.

### CROSS-REFERENCE TO RELATED APPLICATIONS

This application claims priority to U.S. Provisional Patent Application No. 63/383,432, filed Nov. 11, 2022, the entire content of which is incorporated herein by reference in its entirety.

### INTRODUCTION

Embodiments of the present disclosure relate to dynamic user interfaces. More specifically, embodiments of the present disclosure relate to using machine learning to modify user interfaces.

In a wide variety of systems and platforms, efficient and well-designed user interfaces are important for providing and receiving vital information to and from users. Often, it is desired that the interface be able to receive and return information quickly and easily, so as to facilitate other operations and procedures. For example, in healthcare settings, healthcare providers (e.g., nurses, doctors, and other care providers) frequently interact with graphical user interfaces (GUIs) in order to input updated information from patients, monitor ongoing therapies, request or order needed supplies and medications, and the like. However, in conventional systems, these interfaces are generally static, and are nearly always outdated and complex. That is, the interfaces are usually fixed or predefined and the arrangement or content does not generally change adequately to address changing circumstances or context. Similarly, conventional interfaces are often little no more than a set of input fields (e.g., to receive patient information) and a button to enter the data. As a result, the interfaces often fail to adequately serve the underlying purpose in a reliable and efficient manner (such as ensuring that the entered information is accurate, and providing concrete and actionable information to the user).

Improved systems and techniques to provide dynamic user interfaces are needed.

### SUMMARY

According to one embodiment presented in this disclosure, a method is provided. The method includes: receiving user interaction from a user via a graphical user interface (GUI) of a computing device; in response to receiving the user interaction, collecting a set of user data associated with the user; generating a first stress score by processing the set of user data using a stress model; and in response to determining that the first stress score satisfies one or more defined criteria: generating a first prompt for the user, wherein the first prompt requests additional user interaction, as compared to a default prompt; and outputting the first prompt via the GUI.

According to one embodiment presented in this disclosure, a system is provided. The system comprises: a memory comprising computer-executable instructions; and one or more processors configured to execute the computer-executable instructions and cause the processing system to perform an operation comprising: receiving user interaction from a user via a graphical user interface (GUI) of a computing device; in response to receiving the user interaction, collecting a set of user data associated with the user; generating a first stress score by processing the set of user data using a stress model; and in response to determining that the first stress score satisfies one or more defined criteria: generating a first prompt for the user, wherein the first prompt requests additional user interaction, as compared to a default prompt; and outputting the first prompt via the GUI.

According to one embodiment presented in this disclosure, a non-transitory computer-readable medium is provided, comprising computer-executable instructions that, when executed by one or more processors of a processing system, cause the processing system to perform an operation comprising: receiving user interaction from a user via a graphical user interface (GUI) of a computing device; in response to receiving the user interaction, collecting a set of user data associated with the user; generating a first stress score by processing the set of user data using a stress model; and in response to determining that the first stress score satisfies one or more defined criteria: generating a first prompt for the user, wherein the first prompt requests additional user interaction, as compared to a default prompt; and outputting the first prompt via the GUI.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example environment for dynamic interface modification.

FIG. 2 depicts an example workflow to generate user stress models to drive interface modifications.

FIG. 3 depicts an example workflow to use user stress models to drive interface modifications.

FIG. 4 depicts an example dynamic interface with visual output based on a stress model.

FIG. 5 depicts an example dynamic interface requesting further interaction based on a stress model.

FIG. 6 depicts an example dynamic interface requesting textual interaction based on a stress model.

FIG. 7 depicts an example dynamic interface indicating additional information and instruction based on a stress model.

FIG. 8 is a flow diagram depicting an example method for updating interfaces using stress models.

FIG. 9 is a flow diagram depicting an example method for generating a stress model for dynamic interface modification.

FIG. 10 is a flow diagram depicting an example method for refining stress models based on interface feedback.

FIG. 11 is a flow diagram depicting an example method for processing data with a stress model.

FIG. 12 is a flow diagram depicting an example method for modifying interfaces based on patient information and user context.

FIG. 13 is a flow diagram depicting an example method for modifying user interfaces.

FIG. 14 is a flow diagram depicting an example method for outputting dynamic prompts via user interfaces.

FIG. 15 depicts an example computing device configured to perform various aspects of the present disclosure.

Additional aspects of the present disclosure can be found in the attached appendix.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved and dynamic user interfaces. In some embodiments, based on the context of a user interaction, the system can dynamically update or modify a graphical user interface (GUI) to better serve the underlying purpose of the interface. In some aspects, the system can determine or infer the emotional state of the user (e.g., their stress level, whether they are paying full attention or are distracted, and the like), and modify or update the GUI in response to this context. For example, if the determined user stress meets or exceeds defined and/or learned criteria, the system may update the GUI to include additional information, to require additional interaction or response, and the like.

In some embodiments, a variety of user data can be collected and processed using one or more stress models (which may include rules-based models and/or trained machine learning models) to generate a stress score (or some other measure) indicating the predicted stress or attention of the user. In some aspects, the user data can include workload information for the user, such as the number of hours they have been working this shift (e.g., whether they just started or near the end of a ten-hour shift), the time of day (e.g., whether it is a first or second shift), the number of patients under the user's care, the acuity of each patient under the user's care (e.g., generated using machine learning), and the like.

In an aspect, this score or measure can then be used to generate and provide specific interface customizations, such as to require additional interaction from the user, in order to mitigate or prevent any potential harm caused by the elevated stress. For example, rather than simply allowing a user to enter information, the system may prompt the user to confirm the data. Similarly, rather than allowing the user to click a “yes” button, the system may ask the user to manually type “yes” or another word or phrase to ensure they are paying full attention. In some aspects, the system can additionally or alternatively provide additional information or instruction based on the stress level. For example, the system may identify patients that are at particular risk (e.g., due to their current state or condition), and notify or remind the user to remain calm or otherwise exercise professionalism while interacting with these patients.

In embodiments, the dynamic interface modification can improve the probability that the user will actively engage with the information, resulting in a number of significant improvements. For example, aspects of the present disclosure may reduce or prevent harm to patients, as the user is more likely to enter information accurately and fully understand information presented via the interface. In conventional systems, it is common for the user to enter incorrect information (e.g., incorrect heart rate data) and/or miss important pieces of information being output by the interface, particularly when stress levels are high. Such mistakes can cause serious harm to patients.

Similarly, in some aspects, the dynamic interfaces described herein can improve the operations of the computing system, such as by increasing accuracy and reducing computational expense of the system. For example, as the dynamic interface improves the data ingestion, they can prevent the need for additional data preprocessing, verification, or other operations that are needed in conventional systems. Similarly, if the input data is used to train or refine any downstream systems (e.g., to train a machine learning model), the models can have significantly improved accuracy and reliability (e.g., because they are trained using more accurate and reliable data input using dynamically generated interfaces). Moreover, in some embodiments that only selectively modify the interface (e.g., only requesting additional interaction when stress level is high), the present disclosure can provide these and other improvements while minimizing computational expense and latency of generating and using the interfaces.

### Example Environment for Dynamic Interface Modification

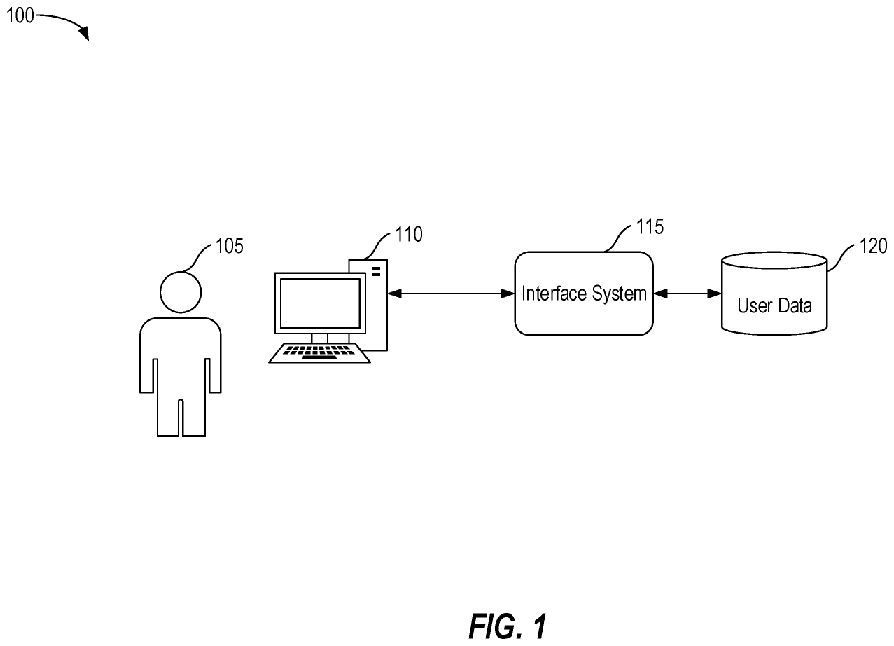

FIG. 1 depicts an example environment 100 for dynamic interface modification.

In the illustrated environment 100, an interface system 115 is communicatively coupled with a user device 110, which is used by one or more users 105. Generally, the user device 110 and interface system 115 may be communicatively linked using one or more wired and/or wireless connections and/or networks. In at least one aspect, the user device 110 and interface system 115 are linked at least in part via the Internet. Although a single user device 110 and interface system 115 are depicted for conceptual clarity, in embodiments, there may be any number of user devices 110 and/or interface systems 115. Additionally, though depicted as discrete components for conceptual clarity, in some aspects, the operations of the interface system 115 may be incorporated within or performed by the user device 110 (and vice versa).

Although depicted as a desktop computer, the user device 110 can generally correspond to any computing device capable of outputting an interface (e.g., a graphical user interface (GUI)) to users 105. For example, the user device 110 may be a smartphone, tablet, laptop, terminal, wearable device (e.g., a smart watch), and the like. In some embodiments, the user device 110 may be used by, assigned to, or otherwise associated with a specific user 105. That is, the user device 110 may be used by a single user 105 (either exclusively or usually), such as a personal laptop. In some embodiments, the user device 110 may be a shared device that can be used by multiple users 105 (simultaneously or sequentially), such as a wall-mounted terminal in a common area.

Generally, the user device 110 can be used to output information to users 105 (via an interface) and/or to receive information from users 105 (via an interface). In embodiments, the user device 110 may be configured to provide the interface (e.g., to output and/or receive information) using any suitable technology. For example, for outputting information, the user device 110 may include one or more visual displays (e.g., screens) to output textual and/or visual information, one or more speakers or other audio devices to output audio, one or more indicators (e.g., lights or tactile indicators) to output information, and the like. For receiving input, the user device 110 may include or use one or more keyboards or other button-based input devices, a computer mouse, one or more touch screens, one or more microphones or other audio devices to receive or record audio input (e.g., voice input), one or more cameras to receive or capture image information (e.g., a picture of the user 105), and the like.

In some aspects, the user 105 is a healthcare provider (e.g., a nurse or caregiver), and the user device 110 is used to retrieve patient information and/or to input new patient information. For example, a healthcare provider may use the user device 110 to retrieve previous diagnoses or clinical notes (e.g., before, during, or after interacting with a patient). Similarly, a healthcare provider may use the user device 110 to record vital statistics such as blood pressure and heart rate, or to record clinical notes or diagnoses (e.g., during or after interacting with a patient). Although some portions of the present disclosure refer to healthcare providers and systems as example, in embodiments, the techniques and systems disclosed herein can be generally applied to any system involving data entry and/or output.

As discussed below in more detail, the interface system 115 is generally used to provide dynamic modification or customization of the interface output by the user device 110. For example, based on the context of an interaction (e.g., the determined or predicted stress level of the user 105), the interface system 115 may modify or generate a GUI that generally improves the operations of the user device 110, improves the outcomes or results of any processes or actions being performed by the user 105 and/or user device 110, improves the outcomes or results of any downstream processes or operations, improves the mood and/or experience of the user, and/or improves the mood, experience, and/or results of other individuals with whom the user is interacting (e.g., patients).

As one example, to improve the functioning of the user device 110, as well as to improve other processes and operations, the interface system 115 may generate a context-driven GUI that provides additional information not otherwise available in conventional systems (e.g., indicating information that is particularly relevant given the current context). As another example, the improved GUI may streamline or reduce barriers to using the interface (e.g., by removing one or more irrelevant or unneeded portions or actions, such as removing a prompt that requires user input), thereby reducing the latency and computational expense of generating, outputting, and using the interface. As yet another example, the improved GUI may allow the user 105 to receive more complete, accurate, or reliable information (e.g., by adding extra prompts to ensure that the user 105 read and understood the information). Similarly, the improved GUI may enable more complete, accurate, or reliable input (e.g., by adding extra prompts to ensure that the user accurately entered the information).

Additionally, in some aspects, the interface system 115 can selectively and dynamically use these modifications only when they are needed (e.g., when user stress is high), thereby providing significant improvements without significant computational expense. These results, in turn, can provide significant improvements for a variety of other operations and systems. For example, the improved GUI may allow the user 105 to recognize and/or change the current context (e.g., to take a deep breath or calm down before proceeding to greet another patient), which not only improves the experience of the user 105 and patient, but can also prevent significant harm to the patient (e.g., preventing or reducing clinical mistakes). Similarly, as the input data may be more reliable or accurate (e.g., because the interface system 115 mitigates or controls for the stress of the user), it can be used readily for downstream operations, such as training machine learning models, in a way that results in improved accuracy (as compared to conventional systems without such controls).

In some embodiments, the interface system 115 can dynamically generate and/or remove interfaces or prompts that require or request additional user interaction, as compared to default prompts or interfaces. For example, during default operations, the interface system 115 may receive input (e.g., blood pressure data), and generate a prompt requesting confirmation if the input is outside of a defined range (e.g., outside of normal blood pressure values). In one aspect, based on the current user context, the interface system 115 may generate a prompt that requires the user to confirm the accuracy of the input information if it is outside of a relatively narrower range, or regardless of whether it is outside the defined range at all. This may allow for improved operations and information accuracy. For example, if the user is stressed or distracted (e.g., by a heavy workload that day), they are generally more prone to making mistakes. By including such additional prompting only during these high-stress times, the interface system 115 can ensure the information is accurate without burdening or adding additional expense or latency during other times.

As another example, during default operations, the interface system 115 may generate a prompt requesting confirmation of input data (e.g., via a “yes” button or “no” button). Based on the current user context, the interface system 115 may generate a prompt that requires additional interaction from the user in order to confirm the accuracy of the input information, such as requiring that they select a specific visual element on the GUI (e.g., a square), manually type their response (e.g., typing “yes” or typing the information a second time), orally confirm the data (e.g., speaking “yes” or speaking the input information), requiring that they scroll or otherwise move to a different interface or portion of the interface before confirming, and the like.

As discussed above and in more detail below, these dynamic and context-specific interfaces that can selectively request or require additional interaction, as compared to the default, can significantly improve the operations of the system.

In the illustrated environment 100, the interface system 115 determines the context, which is used to generate and/or modify the interface, based on user data 120. Although depicted as residing independently from the interface system 115, in some aspects, it can be stored locally within the interface system 115. In some aspects, the user data 120 can include workload information for the user 105 that is using the user device 110. For example, the workload information may include information relating to the current job shift, such as the timing of the shift (e.g., whether it is a first shift (from morning to afternoon), second shift (from afternoon to evening), or third shift (from evening to morning), how long the shift is, how far into the shift the user is (e.g., how many hours have elapsed since the user 105 started the current shift), and type of work being done (e.g., whether the user is interacting directly with patients), and the like.

In at least one embodiment, the user data 120 can include acuity information for the patient(s) being cared for by the user 105. Generally, the acuity can represent the amount of care or effort needed to support a given patient, where higher acuity requires higher levels of care. For example, a patient that needs assistance using the toilet may have higher acuity than a patient that can use the toiler unassisted. In some aspects, the acuity information is generated using one or more trained machine learning models. For example, the interface system 115 or another system may use patient data to train an acuity model, and use the trained model to generate acuity scores or measures for new patient data.

In one embodiment, the acuity model is trained by providing patient information such as their age, weight, diagnoses or disabilities, required care, and the like as input to the model in order to generate an output score. This score can then be compared to a ground-truth (e.g., provided by a healthcare worker) acuity. For example, the score may be compared to a numerical score assigned by the provider, or to a category or classification (e.g., high, medium, or low acuity) defined using score thresholds. Based on the difference between the predicted and actual patient acuity, the model can be iteratively refined to generate more accurate output. In the environment 100, the interface system 115 (or another system) can then determine the acuity of each patient that the user 105 is caring for by processing corresponding patient data using the trained acuity model.

Generally, working with high acuity patients may cause additional stress or tension for the user 105. For example, caring for five patients with relatively high acuity will generally cause more stress, as compared to caring for five patients who have low acuity. In some embodiments, therefore, the interface system 115 can determine or predict the user's stress level based at least in part on the number of patients the user 105 is assisting on the current shift, and/or the assigned acuity scores for each such patient, as reflected in the user data 120.

In some embodiments, the interface system 115 processes the user data 120 using a stress model in order to generate a stress score or measure for the current user 105. This stress model may be a fixed or rules-based model (e.g., specifying weights or contributions of one or more elements of the user data 120 towards stress), or may be a trained machine learning model. For example, the interface system 115 (or another system) may train the model by processing user data 120 (e.g., workload information for the current shift of a user) as input to the stress model in order to generate a stress score (e.g., a numerical score or a classification indicating the predicted level of stress of the user 105, based on their current workload). This score can then be compared against a ground-truth value (e.g., a user-provided indication of how stressed or distracted they currently feel) in order to generate a loss that is used to refine the stress model.

By iteratively performing such training, the stress model learns to generate accurate stress scores for the users. In some aspects, the interface system 115 can use a global stress model for all users 105. That is, a model may be trained and refined using data from multiple users, and this single model may be used to process the user data 120 of whichever user (or users) is currently using the user device 110. In at least one aspect, a personalized or customized model can be trained for each user. For example, a global model may be trained using data from a variety of users, and feedback from each individual user 105 can be used to refine a corresponding model for the specific user. When a user 105 then uses the user device 110, the interface system 115 can identify the user can retrieve the corresponding model, and use this personalized stress model to predict the stress of the user 105.

In some aspects, the user 105 can then provide feedback (e.g., using the interface) to indicate their current level of stress and/or to indicate whether the modification was helpful. This can allow the interface system 115 to further refine the models and GUI generation. For example, if the interface system 115 predicted that the user 105 was highly stressed but the user 105 indicated a low stress level, the interface system 115 may refine the model accordingly (e.g., to generate a lower stress score for the current user data 120). Similarly, if the interface system 115 predicted that the user 105 was not particularly stressed but the user 105 indicates a high level of stress, the interface system 115 may refine the model to generate a higher stress score for the current context.

In some aspects, the user 105 may additionally or alternatively provide feedback on the GUI itself (rather than about their stress level). For example, the feedback may indicate that the generated dynamic GUI was helpful (e.g., providing and/or receiving relevant information in a way that was helpful and useful, or because it reduced the user's stress level), was not helpful (e.g., because it did not include desired information, did not facilitate data entry, and/or increased the user's frustration or stress level). This feedback can allow the interface system 115 to refine its GUI generation procedures.

In at least one aspect, the interface system 115 uses the stress score to generate the GUI based on one or more thresholds. For example, if the stress score is below a first threshold, the interface system 115 may use a default interface. If the score meets or exceeds the first threshold, the interface system 115 may generate an interface requesting more interaction from the user (e.g., to ensure they slow down and fully read and understand the information). In some aspects, if the score meets or exceeds a second (higher) threshold, the interface system 115 may request still more interaction (e.g., typing “yes” rather than simply selecting a button).

In some embodiments, the thresholds can be defined globally or specifically for each user. That is, the interface system 115 may use global stress level thresholds for all users (e.g., where a score greater than a defined value results in the same modifications to the interface for all users), or may use user-specific thresholds (such that different users having the same stress level may receive different interfaces). In at least one aspect, the interface system 115 can refine these thresholds based on user feedback relating to the helpfulness or appropriateness of the interface changes, as discussed above. In this way, the interface system 115 can use machine learning not only to generate the stress scores, but also to generate interfaces based on stress scores (e.g., because the thresholds or other rules are learned and refined based on user feedback).

### Example Workflow to Generate User Stress Models to Drive Interface Modifications

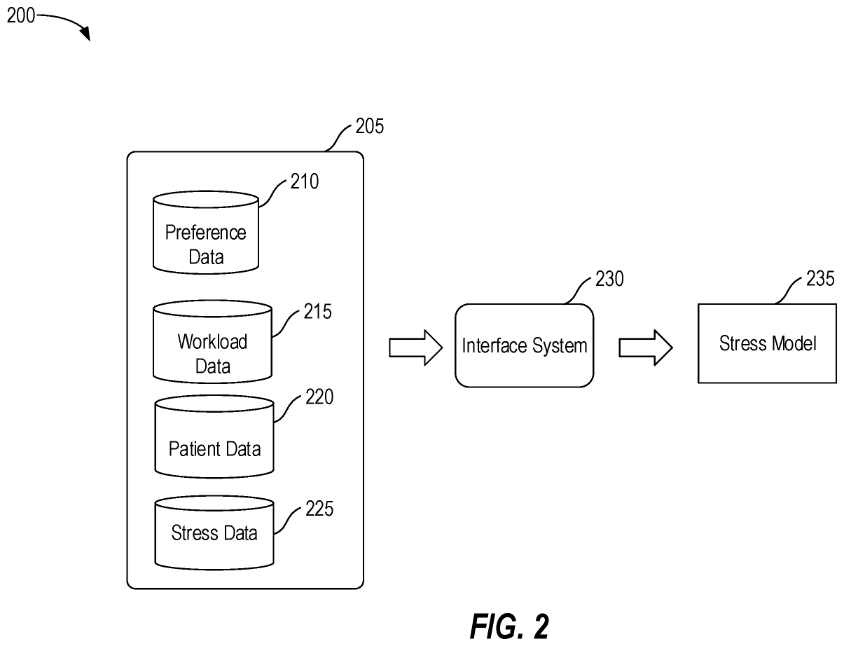

FIG. 2 depicts an example workflow 200 to generate user stress models to drive interface modifications.

In the illustrated workflow 200, a set of historical data 205 is evaluated by an interface system 230 (which may correspond to the interface system 115 of FIG. 1, or may be a discrete system) to generate one or more stress models 235. In embodiments, the interface system 230 may be implemented using hardware, software, or a combination of hardware and software. The historical data 205 (also referred to as user data in some aspects) generally includes data or information associated with one or more users from one or more prior points in time. That is, the historical data 205 may include, for one or more users, a set of one or more snapshots of the user's characteristics or attributes at one or more points in time. For example, the historical data 205 may include attributes for a set of caregivers or other workers in a healthcare setting. In some embodiments, the historical data 205 includes indications of when the data was recorded or relevant (e.g., records indicating which work shift the data corresponds to, and/or what time of day or day of week the data corresponds to). The historical data 205 may generally be stored in any suitable location. For example, the historical data 205 may be stored within the interface system 230, or may be stored in one or more remote repositories, such as in a cloud storage system.

In the illustrated example, the historical data 205 includes, for each user reflected in the data, preference data 210, workload data 215, patient data 220, and stress data 225. In some embodiments, as discussed above, the historical data 205 includes data at multiple points in time for each user. That is, for a given user, the historical data 205 may include multiple sets of workload data 215 (e.g., one set for each shift the user worked), and the like. In some embodiments, the preference data 210, workload data 215, patient data 220, and/or stress data 225 can be linked or associated (e.g., using timestamps or other indications of the relevant time or period) for the data. That is, the interface system 230 may be able to identify the relevant data for any given point or window of time. For example, for a given shift that the user worked, the interface system 230 can identify all the relevant data surrounding this time (e.g., the user's preference data 210 for that shift, the workload data 215 of that shift, the patient data 220 from that shift, and/or the user's stress data 225 for that shift).

In some aspects of the present disclosure, the various components of the historical data 205 are described with reference to a single user for conceptual clarity (e.g., preference data 210 of a single user) and/or for a single time (e.g., a shift). However, it is to be understood that the historical data 205 can generally include such data for any number of users and times.

As discussed in more detail below, the preference data 210 generally corresponds to a set of one or more user-specified preferences. For example, the user may specify interface styles or types of prompt that they prefer, do not prefer, never wish to receive, and the like. In some aspects, the preference data 210 can include information such as feedback from the user (e.g., indicating whether they liked a given interface). In some aspects, the preference data 210 indicates specified or inferred preferences relating to work. For example, the user may prefer to work with older patients, as compared to younger patients. Similarly, the user may prefer to work with patients having one or more particular conditions or disabilities, even if these conditions require substantial effort or care, as compared to one or more other conditions or disabilities. In embodiments, these preferences may help shape the stress of the user, and can therefore be used to define the stress model 235.

In some embodiments, the preference data 210 is curated or selected based on their impact on user stress. For example, in one aspect, an administrator may manually specify attributes or options that have a high impact on user stress, and allow users to enter relevant information for each. In some embodiments, some or all of the features may be inferred or learned (e.g., using one or more feature selection techniques). For example, one or more machine learning models or feature selection algorithms may be used to identify specific preferences (or to determine dynamic weights for each preference) that affect user stress.

As discussed in more detail below, the workload data 215 generally corresponds to information relating to the current workload of the user, such as the number of patients they are caring for, the acuity of each, the duration of the current shift, how long they have been working their current shift (which can be correlated with the reported or inferred stress at the same time during the shift), the time of the shift (e.g., first shift or night shift), and the like.

As discussed in more detail below, the patient data 220 can generally include attributes of any patient(s) that the user is caring for on the current shift and/or cared for on a previous shift. For example, the patient data 220 may additionally or alternatively include the acuity information, as well as other attributes such as their conditions, diagnoses, disabilities, ages, and the like. This information may be relevant for an individual user depending on the particular preferences, as discussed above. In at least one aspect, the patient data 220 can also include information about the outcome(s) experienced by the patient(s) the user is caring for. For example, the patient data 220 may indicate whether any of the patients being cared for by the user recently recovered, declined, or died. These outcomes can similarly have an effect on the stress and mood of the user, and therefore be used to build the stress model 235.

As discussed below in more detail, the stress data 225 can generally include information relating to the level of stress being experienced by the user and/or level of attention they feel at one or more points in time. For example, before, during, and/or after a shift, the user may self-report their stress level and/or attention level. By correlating these reports with the workload data 215 and other information, the interface system 230 may be able to build improved stress models.

In some embodiments, the stress data 225 can include inferred stress. For example, the system may monitor how the user is interacting with a user device, such as to determine how much pressure they are applying to the touchscreen (e.g., where higher pressures indicate higher stress), how quickly they are dismissing prompts or pop-ups (e.g., where quicker dismissal indicates higher stress and/or that they are not reading the prompts), and the like. Additionally, in some aspects, the interface system 230 may use one or more cameras and/or microphones to determine or infer the user's stress level. For example, one or more images of the user may be captured and evaluated (e.g., using trained machine learning models) to identify facial expressions of the user and infer their level of stress. Similarly, audio of the user may be captured and evaluated (e.g., using machine learning) to determine whether they appear stress, such as based on their tone, volume, and/or speed of speech.

Although the illustrated historical data 205 includes several specific components including preference data 210, workload data 215, patient data 220, and stress data 225, in some embodiments, the historical data 205 used by the interface system 230 may include fewer components (e.g., a subset of the illustrated examples) or additional components not depicted. Additionally, though the illustrated example provides general groupings of data to aid understanding, in some embodiments, the historical data 205 may be represented using any number of groups and data structures. For example, the patient data 220 may be reflected in the workload data 215.

As illustrated, the interface system 230 generates one or more stress models 235 based on the historical data 205. The stress model 235 generally specifies a set of weights for the various features or attributes of the historical data 205. In some embodiments, the stress model 235 specifies weights specifically for each individual feature (e.g., for each attribute in the workload data 215). For example, a first attribute such as current time may be associated with a lower weight than a second attribute such as shift length. Similarly, in some embodiments, the stress model 235 may specify different weights depending on the severity of the feature (e.g., depending on the whether the patient acuity is high or low).

In some embodiments, the stress model 235 is a fixed rules-based model. In some embodiments, the stress model 235 is a trained model. In one such embodiment, during a training phase, the interface system 230 may process the historical data 205 (other than the stress data 225) for a given user at a given time as input to the stress model 235 in order to generate a predicted stress score or measure, also referred to in some aspects as a test stress score or measure, (e.g., indicating the amount of stress that the user is likely experiencing, and/or the probability or confidence that the user is experiencing the predicted amount of stress). The interface system 230 can then compare the generated/test stress measure to a ground-truth (e.g., the reported, determined, or inferred stress data 225 that corresponds to the time when the input data was collected or recorded). The difference between the generated and actual scores can be used to refine the weights of the stress model 235, and the model can be iteratively refined (e.g., using data from multiple users and/or multiple points in time) to accurately predict user stress based on their workload and other factors.

In some embodiments, during or after training, the interface system 230 may prune the stress model 235 based in part on the learned weights. For example, if the learned weight for a given feature (e.g., a specific element of the workload data 215) is below some threshold (e.g., within a threshold distance from zero), the interface system 230 may determine that the feature has no impact (or negligible impact) on the stress of users (or of the particular user, in the case of personalized models). Based on this determination, the interface system 230 may cull or remove this feature from the stress model 235 (e.g., by removing one or more neurons, in the case of a neural network). For future evaluations, the interface system 230 need not receive data relating to these removed features (and may refrain from processing or evaluating the data if it is received). In this way, the stress model 235 can be used more efficiently (e.g., with reduced computational expense and latency) to yield accurate evaluations.

In some embodiments, the interface system 230 can generate multiple stress models 235. For example, a separate stress model 235 may be generated for each region (e.g., with a unique model for each country). This may allow the interface system 230 to account for facility-specific, region-specific, or culture-specific changes in stress response (e.g., due to climate, average sunlight, and the like). In other embodiments, the interface system 230 generates a universal stress model 235. In at least one embodiment, a global stress model 235 may use similar considerations (e.g., location, region, and the like) as input features.

In some embodiments, the interface system 230 outputs the stress model 235 to one or more other systems for use. That is, the interface system 230 may distribute the stress model 235 to one or more downstream systems, where each downstream system is used to predict user stress (e.g., to drive interface modification). For example, the interface system 230 may deploy the stress model 235 to one or more servers associated with specific facilities or care providers, and these servers may use the model to evaluate user stress and drive dynamic interface modification. In at least one embodiment, the interface system 230 can itself use the stress model 235 to evaluate service plans.

### Example Workflow to Use User Stress Models to Drive Interface Modifications

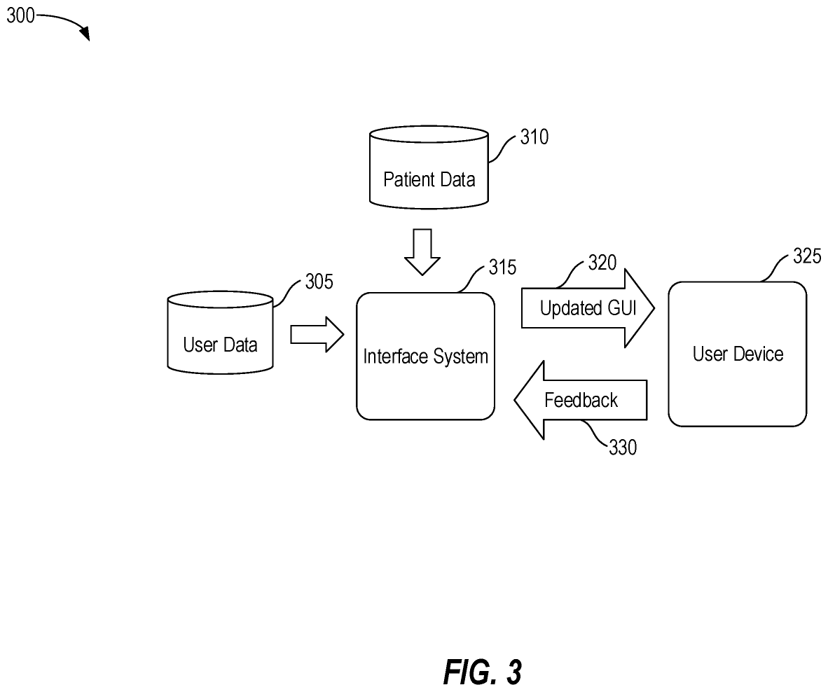

FIG. 3 depicts an example workflow 300 to use user stress models to drive interface modifications.

In the illustrated workflow 300, an interface system 315 (which may correspond to the interface system 115 of FIG. 1, and/or the interface system 230 of FIG. 2) is communicatively coupled with a user device 325 (which may correspond to the user device 110 of FIG. 1). As discussed above, the user device can be used by one or more users (e.g., users 105 of FIG. 1). Generally, the user device 325 and interface system 315 may be communicatively linked using one or more wired and/or wireless connections and/or networks. Additionally, though depicted as discrete components for conceptual clarity, in some aspects, the operations of the interface system 315 may be incorporated within or performed by the user device 325 (and vice versa).

As discussed above, the user device 325 can generally correspond to any computing device capable of outputting an interface (e.g., GUI) to users. For example, the user device 325 may be a smartphone, tablet, laptop, terminal, wearable device (e.g., a smart watch), and the like. In some embodiments, the user device 325 may be used by, assigned to, or otherwise associated with a specific user. That is, the user device 325 may be used by a single user (either exclusively or usually), such as a personal laptop. In some embodiments, the user device 325 may be a shared device that can be used by multiple users (simultaneously or sequentially), such as a wall-mounted terminal in a common area.

In embodiments, the user device 325 may be configured to provide the interface (e.g., to output and/or receive information) using any suitable technology. For example, for outputting information, the user device 325 may include one or more visual displays (e.g., screens) to output textual and/or visual information, one or more speakers or other audio devices to output audio, one or more indicators (e.g., lights or tactile indicators) to output information, and the like. For receiving input, the user device 325 may include or use one or more keyboards or other button-based input device, a computer mouse, one or more touch screens, one or more microphones or other audio devices to receive or record audio input (e.g., voice input), one or more cameras to receive or capture image information (e.g., a picture of the user), and the like.

In some aspects, the user device 325 is used in a healthcare setting (e.g., a hospital, a residential care facility, a therapy center, and the like), as discussed above. For example, the user may be a healthcare provider (e.g., a nurse or caregiver), and the user device 325 may be used to retrieve patient information and/or to input new patient information.

As discussed below in more detail, the interface system 315 is generally used to provide dynamic modification or customization of the interface output by the user device 325. For example, based on the context of an interaction (e.g., the determined or predicted stress level of the user), the interface system 315 may modify or generate a GUI that generally improves the operations of the user device 325, improves the outcomes or results of any processes or actions being performed by the user and/or user device 325, improves the outcomes or results of any downstream processes or operations, improves the mood and/or experience of the user, and/or improves the mood, experience, and/or results of other individuals with whom the user is interacting (e.g., patients).

In the illustrated example, when a user begins using the user device 325 (e.g., interacting with an interface on the user device 325), the interface system 315 can identify the user, and retrieve corresponding user data 305. In embodiments, the interface system 315 or user device 325 can identify the user in a variety of ways. For example, in some aspects, the user device 325 identifies the user, and reports this identity to the interface system 315. In some embodiments, the interface system 315 performs the identification.

Identifying the user can generally include any suitable technique to uniquely identify the user in a way that allows the corresponding user data 305 to be retrieved. This may include identifying the user as an individual (e.g., as John Doe), identifying a user identifier (e.g., User ID 12345), and the like. In some embodiments, the user is identified using login information or other credentials. For example, when using the user device 325, the user may supply credentials (such as a username and password, or by scanning a badge or other authentication device) to enable access to the interface/information. In some embodiments, the user is identified using facial recognition and/or voice recognition (e.g., using one or more trained machine learning models to process a picture of the user and/or a voice recording).

Using the identity of the user, the interface system 315 retrieves the corresponding set of user data 305. In some aspects, as discussed above, the user data 305 can include workload information for the user (e.g., for the current shift they are working at their job). For example, as discussed above, the user data 305 may include information such as the shift type (e.g., first, second, third), the shift length (e.g., whether it is a double shift, or the number of hours the shift lasts), how much time has elapsed since the shift began and/or how much time remains until the shift ends, what the current time is, how many patients they are caring for or have cared for during the shift, information about such patients (such as their identities, locations, acuity scores, and the like), what tasks the user has performed or will perform during the shift (e.g., how many times they assist patients with various activities) and the like. Generally, the user data 305 can include any data that may affect the user's stress levels.

In some embodiments, the user data 305 can additionally or alternatively include data beyond their current workload, such as previous workloads (e.g., during the previous shift or day), user preferences, other events in the user's life (e.g., personal stressors or occurrences, such as moving to a new house), and the like.

In the illustrated example, the interface system 315 can additionally receive or retrieve patient data 310. For example, based on the patient(s) indicated in the user data 305 (e.g., the patients that the user is caring for or has cared for during the current shift), the interface system 315 can identify and retrieve the corresponding patient data 310 for these relevant patients. In some embodiments, as discussed above, the patient data 310 may include information such as the patient outcomes, conditions, diagnoses, disabilities, demographic information, and the like. In at least one aspect, the patient data 310 includes acuity information for each patient. For example, as discussed above, one or more trained or fixed models may be used to process all or a subset of the patient data for a given patient in order to generate an acuity score representing the amount of care they require. These acuity scores can then be provided to the interface system 315.

In an embodiment, as discussed above, the interface system 315 can use the user data 305 and/or patient data 310 to generate stress score or measure for the user, and use the stress score to generate an updated GUI 320, which is then output by the user device 325. In some embodiments, as discussed above, the interface system 315 can use a trained stress model to predict or infer the level of stress the user is experiencing by processing the user data 305 and/or patient data 310 using a stress model. In some aspects, as discussed above, the stress model can be a global model (or a model that is otherwise used for multiple users). In at least one aspect, the interface system 315 uses a personalized model (e.g., a model that has been trained or refined using data specific to the current user).

In some embodiments, the interface system 315 uses the stress score to generate the updated GUI 320 based on one or more thresholds. For example, if the stress score is below a first threshold, the interface system 315 may use a default interface. If the score meets or exceeds the first threshold, the interface system 315 may generate an interface requesting more interaction from the user (e.g., to ensure they slow down and fully read and understand the information). In some aspects, if the score meets or exceeds a second (higher) threshold, the interface system 315 may request still more interaction (e.g., typing “yes” rather than simply selecting a button).

In some embodiments, the thresholds can be defined globally or specifically for each user. That is, the interface system 315 may use global stress level thresholds for all users (e.g., where a score greater than a defined value results in the same modifications to the interface for all users), or may use user-specific thresholds (such that different users having the same stress level may receive different interfaces). In at least one aspect, the interface system 315 can refine these thresholds based on user feedback relating to the helpfulness or appropriateness of the interface changes, as discussed above. In this way, the interface system 315 can use machine learning not only to generate the stress scores, but also to generate interfaces based on stress scores (e.g., because the thresholds or other rules are learned using user feedback).

In some embodiments, to generate the updated GUI 320, the interface system 315 can dynamically generate and/or remove interfaces or prompts that require or request additional user interaction, as compared to default prompts or interfaces. For example, during default operations, the user device 325 may receive input (e.g., blood pressure data), and generate a prompt requesting confirmation if the input is outside of a defined range (e.g., outside of normal blood pressure values). In one aspect, based on the current user context, the interface system 315 may generate an updated GUI 320 that requires the user to confirm the accuracy of the input information if it is outside of a relatively narrower range, or regardless of whether it is outside the defined range at all. This may allow for improved operations and information accuracy. For example, if the user is stressed or distracted (e.g., by a heavy workload that day), they are generally more prone to making mistakes. By including such additional prompting only during these high-stress times, the interface system 315 can ensure the information is accurate without burdening or adding additional expense or latency during other times.

As another example, during default operations, the interface system 315 may generate an interface requesting confirmation of input data (e.g., via a “yes” button or “no” button). Based on the current user context, the interface system 315 may generate an updated GUI 320 that requires additional interaction from the user in order to confirm the accuracy of the input information, such as requiring that they select a specific visual element on the GUI (e.g., a square), manually type their response (e.g., typing “yes” or typing the information a second time), orally confirm the data (e.g., speaking “yes” or speaking the input information), requiring that they scroll or otherwise move to a different interface or portion of the interface before confirming, and the like.

In the illustrated example, the user can then (via the user device 325) optionally provide feedback 330 to the interface system 315. As discussed above, the feedback can generally indicate information such as whether the predicted stress level is accurate, what the user's current stress level is, whether the updated GUI 320 was helpful, useful, or appropriate, and the like. This can allow the interface system 315 to refine the models and GUI generation. For example, if the interface system 315 predicted that the user was highly stressed but the user indicated a low stress level, the interface system 315 may refine the stress model accordingly (e.g., to generate a lower stress score for the current user data 305).

In some aspects, feedback 330 relating to the GUI itself (rather than about the user stress level) can be used to refine the GUI creation process. For example, if the feedback indicates that the updated GUI 320 was not appropriate or helpful, the interface system 315 may refine the GUI generation model (e.g., modifying one or more stress thresholds that trigger various modifications) to generate a better-updated GUI 320 for future use. In at least one embodiment, the interface system 315 can additionally or alternatively add and/or remove modification options based on the feedback 330. For example, if the user indicates that a given modification (e.g., requiring that they type “yes”) is never helpful and/or always adds to their stress, the interface system 315 may determine to refrain from using such a modification for future updated GUIs 320 generated for the specific user.

In at least one embodiment, the feedback 330 can additionally or alternatively include passively collected information (e.g., information about the interaction that does not require user input). For example, the feedback 330 may indicate how much time elapsed from when a prompt was output to when it was dismissed or cleared, how much pressure the user is applying to a touchscreen or button, the facial expression and/or verbal cues given by the user (e.g., an exasperated sigh), and the like. This feedback 330 can similarly be used to determine whether the updated GUI was appropriate.

In some embodiments, based on the feedback 330, the interface system 315 can generate another updated GUI 320 for the user. For example, if the feedback 330 indicates that the GUI is not appropriate, the interface system 315 can immediately return an updated GUI 320 that differs. As another example, if the feedback 330 indicates that the user still dismissed the prompt too quickly, the interface system 315 can generate an updated GUI 320 requiring a relatively higher level of interaction (e.g., requesting that the user type a specific phrase in order to dismiss the prompt) and/or providing an additional reminder to be thorough and vigilant.

As illustrated, the workflow 300 can thereby repeat any number of times, enabling the interface system 315 to continue to provide improved interfaces to user devices.

### Example Dynamic Interface with Visual Output Based on a Stress Model

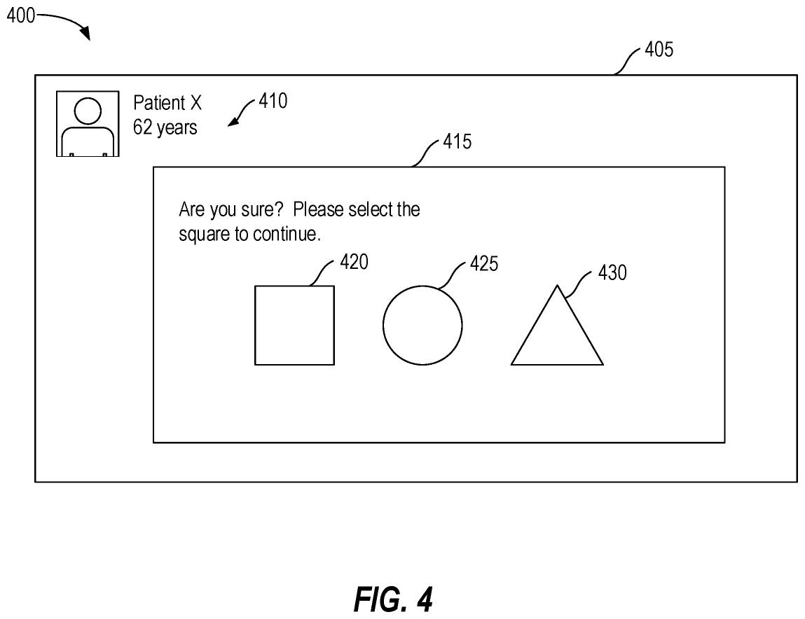

FIG. 4 depicts an example dynamic interface 400 with visual output based on a stress model.

In some embodiments, the interface 400 is generated by an interface system, such as the interface system 115 of FIG. 1, the interface system 230 of FIG. 2, and/or the interface system 315 of FIG. 3. The interface 400 may be output (e.g., via a screen or display) by a user device, such as the user device 110 of FIG. 1 and/or the user device 325 of FIG. 3. In the illustrated example, the interface 400 is generated based on the context of the user to whom the interface 400 is being provided. For example, based on the user's data (e.g., current workload), the interface system can generate a stress measure, and use this stress measure to generate a dynamic interface 400 that is better suited for the current context.

In the illustrated example, the interface 400 is outputting a patient profile 405. The patient profile 405 includes a first portion 410 for displaying and/or receiving patient information, such as their name, age, and picture. In the illustrated example, the interface 400 also includes a prompt 415, requesting interaction from the user prior to allowing them to proceed interacting with the patient profile 405. In one embodiment, during default or ordinary operations (e.g., when the user stress is below a defined threshold), the interface system may use a default interface that does not include the prompt 415, or includes a prompt that requires less interaction. For example, the interface system may simply output the patient profile 405 without further interaction, or may request that the user click an “OK” button before proceeding.

In some embodiments, the prompt 415 is used prior to allowing the user to continue viewing the patient profile 405 or before viewing additional information (e.g., condition data), and/or before allowing the user to enter, record, or save updated data or information to the patient profile 405. For example, when the user selects, requests, or otherwise opens the patient profile 405, the interface system may generate the interface 400 to request that the user confirm that the provided profile corresponds to the patient that the user is actually interacting with or actually intended to select. This can ensure that the user is, indeed, reviewing or modifying the correct patient profile 405, thereby significantly increasing the accuracy of the data and reducing patient harm. For example, in conventional systems, it is common for users (particularly when under stress) to mistakenly retrieve the incorrect profile. They then review inaccurate information for the patient they are actually interacting with, and may make further inappropriate or inaccurate decisions or assumptions based on this erroneous information.

Similarly, if the prompt 415 is provided prior to allowing the user to input new information, the interface system can use it to ensure that the user has entered accurate information. For example, in conventional systems, it is common for users (particularly when under stress) to mistakenly record information under the wrong profile or chart, or to enter incorrect information (e.g., the wrong blood pressure). This can cause significant confusion, as well as harm to the patients. Further, if the data is used for any subsequent operations (such as training a machine learning model to predict various patient attributes, or to predict the efficacy of a given treatment based on the patient attributes), entering information under the wrong profile will result in erroneous training data. As a result, the trained models will suffer from reduced accuracy and reliability.

In the illustrated example, the additional interaction requested by the prompt 415 is to select a specific visual element (e.g., a shape) in the interface 400. Specifically, the prompt 415 includes a square 420, a circle 425, and a triangle 430, and asks the user to select the square 420. In some aspects, the specific indicated element may be selected randomly or pseudo-randomly, such that a different object is indicated each time the prompt 415 is output. This prompt 415 may thereby require that the user take the time to actually read the prompt 415 before dismissing it. Although the illustrated example depicts shapes as the visual components, in some aspects, other data can be used, such as pictures of patients (where the prompt 415 requests that the user select the picture of the patient that corresponds to the patient profile 405).

In an embodiment, if the user fails to perform the requested interaction (e.g., if the user selects the wrong shape), the interface system can take a variety of actions, including generating a new prompt 415 (indicating a different shape), outputting a reminder to read the information carefully before proceeding, generating a new prompt requesting a higher level of interaction, and the like.

Once the user performs the requested interaction, the interface 400 can be updated accordingly (e.g., by removing the prompt 415 and performing the requested action, such as outputting the patient profile 405, saving the input information, and the like.

In this way, the dynamic interface 400 can significantly improve the operations of the user device (e.g., requiring additional prompts or interaction only during stressful times, as compared to using them for all interactions), improve data accuracy (e.g., ensuring that the input data is accurate and reliable), reduce potential harm to patients (e.g., preventing the data from being associated with the incorrect patient), and the like.

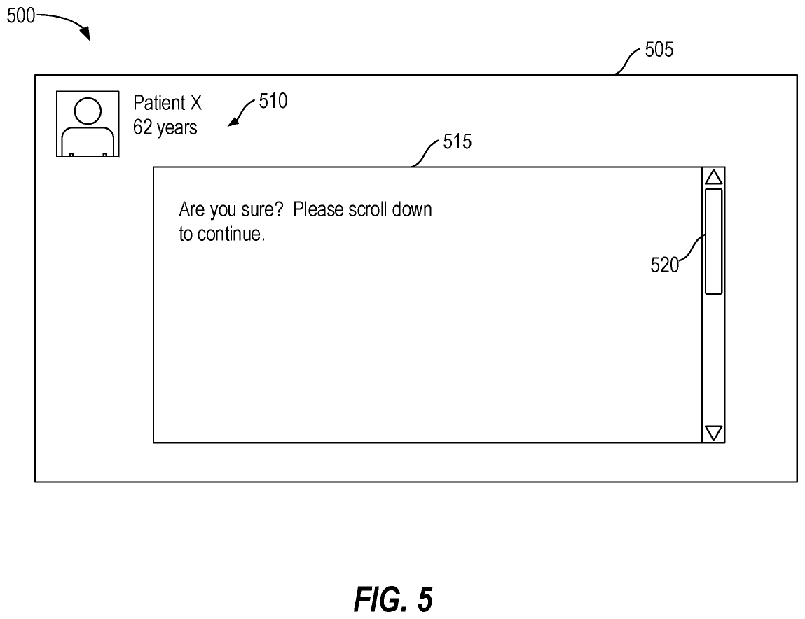

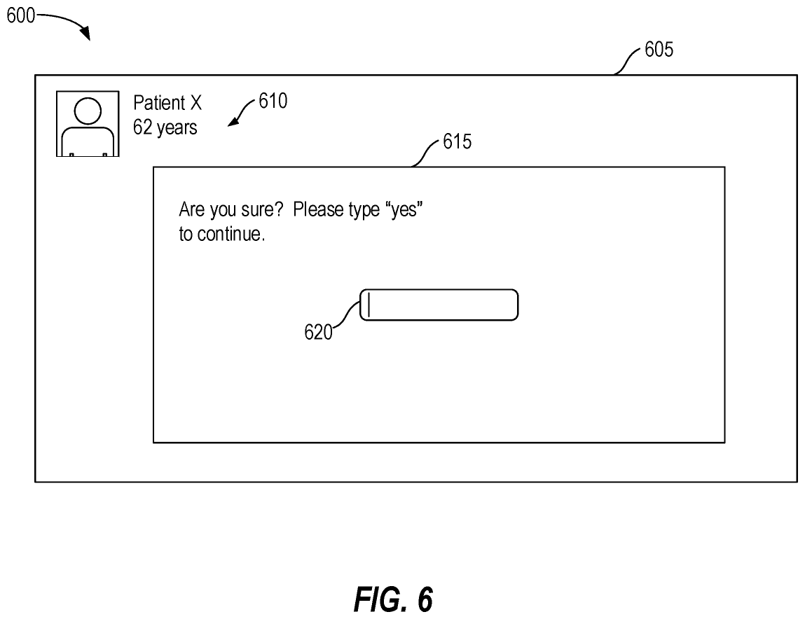

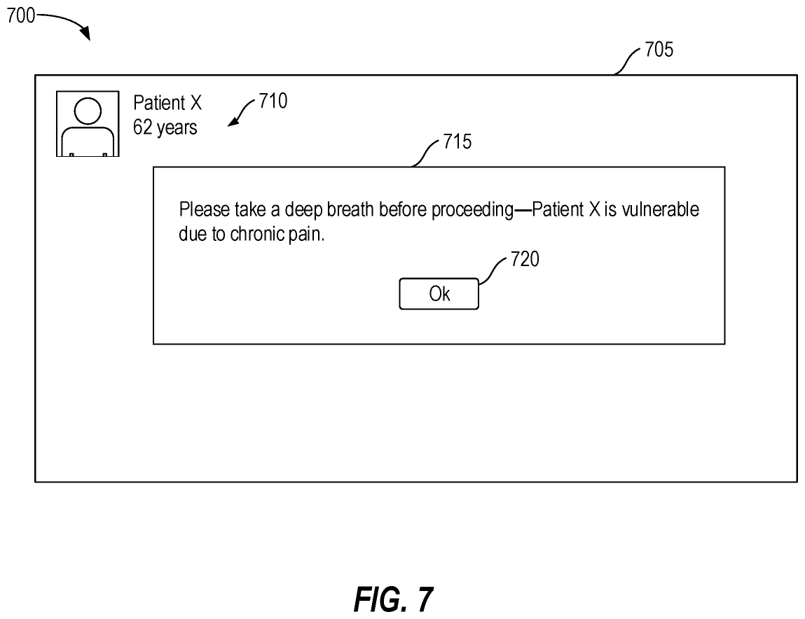

The specific interface 400 is intended as one example interaction and prompt that the interface system can generate. However, the specific interactions and prompts used may vary depending on the particular implementation. Additionally, in some embodiments, the interface system may use a combination of modifications or prompts, such as combining the prompt 415 with the prompt 515 of FIG. 5, the prompt 615 of FIG. 6, and/or the prompt 715 of FIG. 7, in order to improve the system functionality.

### Example Dynamic Interface Requesting Further Interaction Based on a Stress Model

FIG. 5 depicts an example dynamic interface 500 requesting further interaction based on a stress model.

In some embodiments, the interface 500 is generated by an interface system, such as the interface system 115 of FIG. 1, the interface system 230 of FIG. 2, and/or the interface system 315 of FIG. 3. The interface 500 may be output (e.g., via a screen or display) by a user device, such as the user device 110 of FIG. 1 and/or the user device 325 of FIG. 3. In the illustrated example, the interface 500 is generated based on the context of the user to whom the interface 500 is being provided. For example, based on the user's data (e.g., current workload), the interface system can generate a stress measure, and use this stress measure to generate a dynamic interface 500 that is better suited for the current context.

In the illustrated example, the interface 500 is outputting a patient profile 505. The patient profile 505 includes a first portion 510 for displaying and/or receiving patient information, such as their name, age, and picture. In the illustrated example, the interface 500 also includes a prompt 515, requesting interaction from the user prior to allowing them to proceed interacting with the patient profile 505. In one embodiment, during default or ordinary operations (e.g., when the user stress is below a defined threshold), the interface system may use a default interface that does not include the prompt 515, or includes a prompt that requires less interaction. For example, the interface system may simply output the patient profile 505 without further interaction, or may request that the user click an “OK” button before proceeding.

In some embodiments, the prompt 515 is used prior to allowing the user to continue viewing the patient profile 505 or before viewing additional information (e.g., condition data), and/or before allowing the user to enter, record, or save updated data or information to the patient profile 505. For example, when the user selects, requests, or otherwise opens the patient profile 505, the interface system may generate the interface 500 to request that the user confirm that the provided profile corresponds to the patient that the user is actually interacting with or actually intended to select. This can ensure that the user is, indeed, reviewing or modifying the correct patient profile 505, thereby significantly increasing the accuracy of the data and reducing patient harm. For example, in conventional systems, it is common for users (particularly when under stress) to mistakenly retrieve the incorrect profile. They then review inaccurate information for the patient they are actually interacting with, and may make further inappropriate or inaccurate decisions or assumptions based on this erroneous information.

Similarly, if the prompt 515 is provided prior to allowing the user to input new information, the interface system can use it to ensure that the user has entered accurate information. For example, in conventional systems, it is common for users (particularly when under stress) to mistakenly record information under the wrong profile or chart, or to enter incorrect information (e.g., the wrong blood pressure). This can cause significant confusion, as well as harm to the patients. Further, if the data is used for any subsequent operations (such as training a machine learning model to predict various patient attributes, or to predict the efficacy of a given treatment based on the patient attributes), entering information under the wrong profile will result in erroneous training data. As a result, the trained models will suffer from reduced accuracy and reliability.

In the illustrated example, the additional interaction requested by the prompt 515 is to scroll the prompt (using scroll bar 520) or otherwise move the screen output to a different portion of the prompt 515 or interface 500. Specifically, the prompt 515 includes a scroll bar 520, and asks the user to scroll down to continue. In some aspects, the length of the scroll bar 520 may be generated randomly or pseudo-randomly, or may be generated based at least in part on the generated stress level (e.g., where higher stress levels result in longer scroll bars). This prompt 515 may thereby require that the user take the time to actually read the prompt 515 and perform the requested action before dismissing it. Although the illustrated example depicts a scroll bar 520 as the requested action, in some aspects, other actions can be used, such as moving the prompt or a portion of it to a specified portion of the interface 500 (e.g., using a mouse), or clicking and dragging to a different portion of the prompt 515.

In some aspects, after scrolling down, the prompt 515 may be automatically dismissed by the device, and/or the user may be requested to perform a further action (such as clicking a button or visual element). In at least one embodiment, whether further interaction is required can be determined based on the stress level of the user, where higher stress levels may require higher interactions. Further, although not included in the illustrated example, in at least one embodiment, the prompt 515 may include more information or instruction while the user is scrolling, such as suggesting that they use the time it takes to scroll down to take a deep breath and relax.

In an embodiment, if the user fails to perform the requested interaction (e.g., if the user does not scroll down), the interface system can take a variety of actions, including generating a new prompt 515, outputting a reminder to read the information carefully before proceeding, generating a new prompt requesting a higher level of interaction (e.g., requiring a different interaction after scrolling), and the like.

Once the user performs the requested interaction, the interface 500 can be updated accordingly (e.g., by removing the prompt 515 and performing the requested action, such as outputting the patient profile 505, saving the input information, and the like.

In this way, the dynamic interface 500 can significantly improve the operations of the user device (e.g., requiring additional prompts or interaction only during stressful times, as compared to using them for all interactions), improve data accuracy (e.g., ensuring that the input data is accurate and reliable), reduce potential harm to patients (e.g., preventing the data from being associated with the incorrect patient), and the like.

The specific interface 500 is intended as one example interaction and prompt that the interface system can generate. However, the specific interactions and prompts used may vary depending on the particular implementation. Additionally, in some embodiments, the interface system may use a combination of modifications or prompts, such as combining the prompt 515 with the prompt 415 of FIG. 4, the prompt 615 of FIG. 6, and/or the prompt 715 of FIG. 7, in order to improve the system functionality.

### Example Dynamic Interface Requesting Textual Interaction Based on a Stress Model

FIG. 6 depicts an example dynamic interface 600 requesting textual interaction based on a stress model.

In some embodiments, the interface 600 is generated by an interface system, such as the interface system 115 of FIG. 1, the interface system 230 of FIG. 2, and/or the interface system 315 of FIG. 3. The interface 600 may be output (e.g., via a screen or display) by a user device, such as the user device 110 of FIG. 1 and/or the user device 325 of FIG. 3. In the illustrated example, the interface 600 is generated based on the context of the user to whom the interface 600 is being provided. For example, based on the user's data (e.g., current workload), the interface system can generate a stress measure, and use this stress measure to generate a dynamic interface 600 that is better suited for the current context.

In the illustrated example, the interface 600 is outputting a patient profile 605. The patient profile 605 includes a first portion 610 for displaying and/or receiving patient information, such as their name, age, and picture. In the illustrated example, the interface 600 also includes a prompt 615, requesting interaction from the user prior to allowing them to proceed interacting with the patient profile 605. In one embodiment, during default or ordinary operations (e.g., when the user stress is below a defined threshold), the interface system may use a default interface that does not include the prompt 615, or includes a prompt that requires less interaction. For example, the interface system may simply output the patient profile 605 without further interaction, or may request that the user click an “OK” button before proceeding.

In some embodiments, the prompt 615 is used prior to allowing the user to continue viewing the patient profile 605 or before viewing additional information (e.g., condition data), and/or before allowing the user to enter, record, or save updated data or information to the patient profile 605. For example, when the user selects, requests, or otherwise opens the patient profile 605, the interface system may generate the interface 600 to request that the user confirm that the provided profile corresponds to the patient that the user is actually interacting with or actually intended to select. This can ensure that the user is, indeed, reviewing or modifying the correct patient profile 605, thereby significantly increasing the accuracy of the data and reducing patient harm. For example, in conventional systems, it is common for users (particularly when under stress) to mistakenly retrieve the incorrect profile. They then review inaccurate information for the patient they are actually interacting with, and may make further inappropriate or inaccurate decisions or assumptions based on this erroneous information.

Similarly, if the prompt 615 is provided prior to allowing the user to input new information, the interface system can use it to ensure that the user has entered accurate information. For example, in conventional systems, it is common for users (particularly when under stress) to mistakenly record information under the wrong profile or chart, or to enter incorrect information (e.g., the wrong blood pressure). This can cause significant confusion, as well as harm to the patients. Further, if the data is used for any subsequent operations (such as training a machine learning model to predict various patient attributes, or to predict the efficacy of a given treatment based on the patient attributes), entering information under the wrong profile will result in erroneous training data. As a result, the trained models will suffer from reduced accuracy and reliability.

In the illustrated example, the additional interaction requested by the prompt 615 is to type a specific phrase or word (e.g., “yes”) in the interface 600. Specifically, the prompt 615 includes a text entry box 620, and asks the user to type “yes” to proceed. In some aspects, the specific word or phrase may be selected randomly or pseudo-randomly, such that a different word is indicated each time the prompt 615 is output. This prompt 615 may thereby require that the user take the time to actually read the prompt 615 before dismissing it. Although the illustrated example depicts “yes” as the input text, in some aspects, other input text can be used, such as requesting that the user enter their own names, enter the name of the patient, and the like.

In an embodiment, if the user fails to perform the requested interaction (e.g., if the user types the wrong word, misspells the text, or does not enter text for a defined period of time), the interface system can take a variety of actions, including generating a new prompt 615 (requesting that the user type the same string or a different string), outputting a reminder to read the information carefully before proceeding, generating a new prompt requesting a higher level of interaction, and the like.

Once the user performs the requested interaction, the interface 600 can be updated accordingly (e.g., by removing the prompt 615 and performing the requested action, such as outputting the patient profile 605, saving the input information, and the like.

In this way, the dynamic interface 600 can significantly improve the operations of the user device (e.g., requiring additional prompts or interaction only during stressful times, as compared to using them for all interactions), improve data accuracy (e.g., ensuring that the input data is accurate and reliable), reduce potential harm to patients (e.g., preventing the data from being associated with the incorrect patient), and the like.

The specific interface 600 is intended as one example interaction and prompt that the interface system can generate. However, the specific interactions and prompts used may vary depending on the particular implementation. Additionally, in some embodiments, the interface system may use a combination of modifications or prompts, such as combining the prompt 615 with the prompt 415 of FIG. 4, the prompt 515 of FIG. 5, and/or the prompt 715 of FIG. 7, in order to improve the system functionality.

### Example Dynamic Interface Indicating Additional Information and Instruction Based on a Stress Model

FIG. 7 depicts an example dynamic interface 700 indicating additional information and instruction based on a stress model.

In some embodiments, the interface 700 is generated by an interface system, such as the interface system 115 of FIG. 1, the interface system 230 of FIG. 2, and/or the interface system 315 of FIG. 3. The interface 700 may be output (e.g., via a screen or display) by a user device, such as the user device 110 of FIG. 1 and/or the user device 325 of FIG. 3. In the illustrated example, the interface 700 is generated based on the context of the user to whom the interface 700 is being provided. For example, based on the user's data (e.g., current workload), the interface system can generate a stress measure, and use this stress measure to generate a dynamic interface 700 that is better suited for the current context.

In the illustrated example, the interface 700 is outputting a patient profile 705. The patient profile 705 includes a first portion 710 for displaying and/or receiving patient information, such as their name, age, and picture. In the illustrated example, the interface 700 also includes a prompt 715, requesting interaction from the user prior to allowing them to proceed interacting with the patient profile 705. In one embodiment, during default or ordinary operations (e.g., when the user stress is below a defined threshold), the interface system may use a default interface that does not include the prompt 715, or includes a prompt that requires less interaction. For example, the interface system may simply output the patient profile 705 without further interaction, or may request that the user click an “OK” button before proceeding.

In some embodiments, the prompt 715 is used prior to allowing the user to continue viewing the patient profile 705 or before viewing additional information (e.g., condition data), and/or before allowing the user to enter, record, or save updated data or information to the patient profile 705. For example, when the user selects, requests, or otherwise opens the patient profile 705, the interface system may generate the interface 700 to request that the user confirm that the provided profile corresponds to the patient that the user is actually interacting with or actually intended to select. This can ensure that the user is, indeed, reviewing or modifying the correct patient profile 705, thereby significantly increasing the accuracy of the data and reducing patient harm. For example, in conventional systems, it is common for users (particularly when under stress) to mistakenly retrieve the incorrect profile. They then review inaccurate information for the patient they are actually interacting with, and may make further inappropriate or inaccurate decisions or assumptions based on this erroneous information.

Similarly, if the prompt 715 is provided prior to allowing the user to input new information, the interface system can use it to ensure that the user has entered accurate information. For example, in conventional systems, it is common for users (particularly when under stress) to mistakenly record information under the wrong profile or chart, or to enter incorrect information (e.g., the wrong blood pressure). This can cause significant confusion, as well as harm to the patients. Further, if the data is used for any subsequent operations (such as training a machine learning model to predict various patient attributes, or to predict the efficacy of a given treatment based on the patient attributes), entering information under the wrong profile will result in erroneous training data. As a result, the trained models will suffer from reduced accuracy and reliability.

In the illustrated example, the additional interaction requested by the prompt 715 is to pause and take a deep breath before proceeding. Specifically, the prompt 715 includes text instructing or suggesting that the user take a deep breath, as well as indicating that the patient corresponding to the patient profile 705 is in a vulnerable state. In the illustrated example, the prompt 715 further indicates why the patient is at-risk (because they are suffering from chronic pain). This prompt 715 may thereby cause the user take the time to actually read the prompt 715 and ensure they are in a good mental state before dismissing it.

In some aspects, the interface system can include the warning about one or more patient's vulnerability due, in part, to the user's stress level. For example, if the user stress exceeds some threshold, the interface system may determine that they are at particular risk of making mistakes or acting impulsively or with reduced care. Based on this determination, the interface system can evaluate the user data and/or patient records to determine whether any of the user's patients are at particular risk. For example, the interface system may determine whether any of the patients have one or more conditions from a defined list of conditions that make the patient “at risk” or “vulnerable.” If so, the interface system may output an indication of the at-risk patient, as well as a reason for the risk, allowing the user time to review and consider how to best proceed before they interact with the patient(s). For example, the user may decide to take an extra minute to calm down before entering the patient's room, or may otherwise decide to act more warmly and provide more care than they otherwise would.

In some aspects, if no patients are found to be at particular risk, the interface system can nevertheless include an instruction or suggestion to take a breath, meditate, or otherwise calm down before proceeding. Additionally, though the illustrated example depicts a warning relating to a single patient (e.g., the patient corresponding to the patient profile 705), in some aspects, the interface system may output indications of several patients (e.g., the next few at-risk patients that the user is scheduled to interact with).

In an embodiment, if the user fails to perform the requested interaction (e.g., if the user dismisses the prompt within a defined period, without reading it), the interface system can take a variety of actions, including generating a new prompt 715 (e.g., requiring a set amount of time pass before it can be dismissed), outputting a reminder to read the information carefully before proceeding, generating a new prompt requesting a higher level of interaction, and the like.

Once the user performs the requested interaction, the interface 700 can be updated accordingly (e.g., by removing the prompt 715 and performing the requested action, such as outputting the patient profile 705, saving the input information, and the like.

In this way, the dynamic interface 700 can significantly improve the operations of the user device (e.g., requiring additional prompts or interaction only during stressful times, as compared to using them for all interactions), improve data accuracy (e.g., ensuring that the input data is accurate and reliable), reduce potential harm to patients (e.g., preventing the data from being associated with the incorrect patient), and the like.

The specific interface 700 is intended as one example interaction and prompt that the interface system can generate. However, the specific interactions and prompts used may vary depending on the particular implementation. Additionally, in some embodiments, the interface system may use a combination of modifications or prompts, such as combining the prompt 715 with the prompt 415 of FIG. 4, the prompt 515 of FIG. 5, and/or the prompt 615 of FIG. 6, in order to improve the system functionality.

### Example Method for Updating Interfaces Using Stress Models

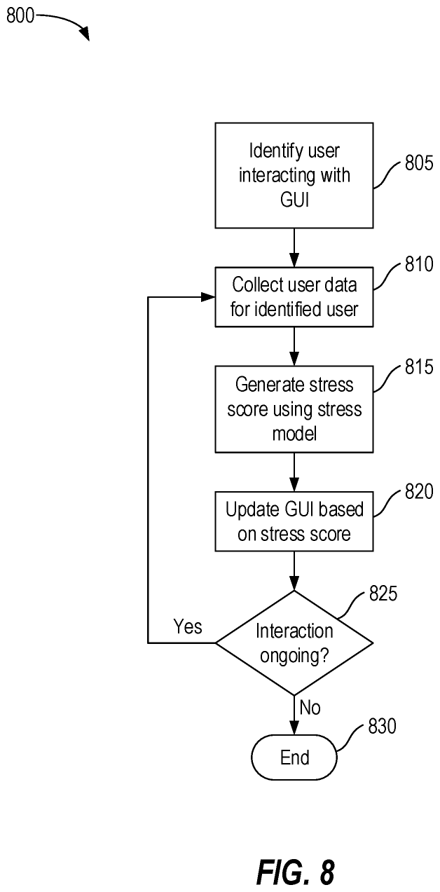

FIG. 8 is a flow diagram depicting an example method 800 for updating interfaces using stress models. In some embodiments, the method 800 is performed by an interface system, such as the interface system 115 of FIG. 1, the interface system 230 of FIG. 2, and/or the interface system 315 of FIG. 3. In some embodiments, the method 800 is performed by a user device (such as the user device 110 of FIG. 1 and/or the user device 325 of FIG. 3), or by an interface system integrated into a user device.

At block 805, the interface system identifies a user that is currently interacting with a GUI or other interface of a user device. For example, as discussed above, the interface system (or user device) may receive user credentials or authentication, use facial recognition, voice recognition, and/or biometric recognition (e.g., fingerprint authentication), and the like in order to identify the user that is interacting with (or desires to interact with) the interface. As discussed above, this identification may include identifying the user individually (e.g., as John Smith), identifying the user's identifier or username, and the like.

At block 810, the interface system collects, retrieves, or receives user data for the identified user (e.g., user data 120 of FIG. 1, and/or user data 305 of FIG. 3). For example, as discussed above, the interface system may access one or more data stores to retrieve information such as the user's current, future, and/or historic workload (e.g., the number of patients they are caring for), the user's preferences, and the like.

At block 815, the interface system generates a stress score, for the identified user, by processing the collected user data using a stress model (e.g., stress model 235 of FIG. 2). In some embodiments, as discussed above, the stress model is a trained machine learning model. This may include a global model used for multiple users (e.g., used to predict stress for all users globally, for all users in a given region, for all users working in a given facility, and the like). In some aspects, the interface system uses a personalized or customized stress model for the user. That is, the interface system may retrieve a model that has been fine-tuned or refined using data specific to the identified user, allowing for improved stress score generation.

The stress score generally indicates the predicted level of stress for the user, and/or the probability that the user's stress meets or exceeds one or more defined thresholds. As discussed above, user data such as the number of patients the user is caring for, the acuity of each such patient, how well matched the user and patients are (e.g., whether they have conflicting or complementary personalities), and the like can all affect the stress of the user. In turn, this stress can affect the user's work. For example, users experiencing higher stress may be more likely to be careless, to make mistakes, to act recklessly or negligently towards patients, and the like.

At block 820, the interface system updates the GUI being used by the user based at least in part on the generated stress score. For example, as discussed above, the interface system may determine whether the stress score exceeds one or more thresholds (which may be fixed, or may be customizable or learnable for the specific user or for a set of users), and generates various prompts (e.g., requiring increasing amounts of interaction or time, as the stress level increases) to output via the GUI. As discussed above, these GUI modifications can ensure that the user takes the time to actively read and understand the information, and significantly reduces the risk of mistakes or harm to the patients.

At block 825, the interface system determines whether the interaction is still ongoing. That is, the interface system can determine whether the user is still interacting with the GUI. If not, the method 800 terminates at block 830. If the interaction is ongoing, the method 800 returns to block 810 to collet new user data. In this way, the interface system can continue to monitor the user's interactions and stress, updating the GUI accordingly, to ensure that the interaction remains efficient, reliable, and accurate.

### Example Method for Generating a Stress Model for Dynamic Interface Modification

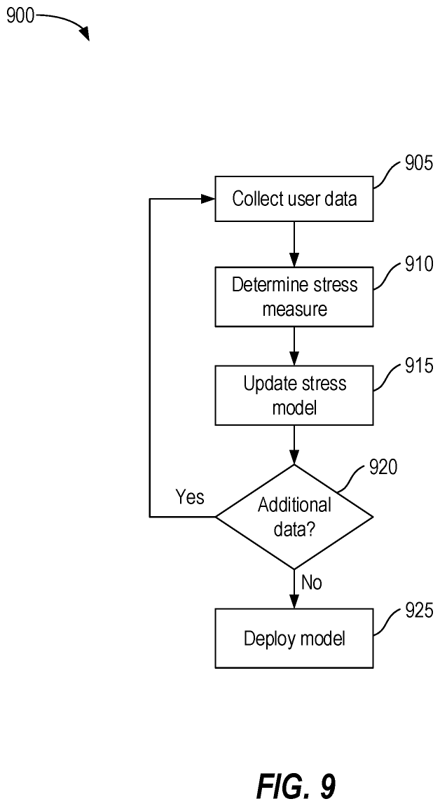

FIG. 9 is a flow diagram depicting an example method 900 for generating a stress model for dynamic interface modification. In some embodiments, the method 900 is performed by an interface system, such as the interface system 115 of FIG. 1, the interface system 230 of FIG. 2, and/or the interface system 315 of FIG. 3. In some embodiments, the method 900 is performed by a user device (such as the user device 110 of FIG. 1 and/or the user device 325 of FIG. 3), or by an interface system integrated into a user device.

At block 905, the interface system collects user data (e.g., historical data 205 of FIG. 2). For example, as discussed above, the interface system may collect, receive, or retrieve user data from one or more prior times (e.g., from prior work shifts). In some embodiments, the interface system collects information for multiple users and/or multiple shifts or times. For example, the interface system may, for each respective user that worked a prior shift, determine the respective workload of the user. Similarly, the interface system may, for each prior shift that was worked by a given user, determine the respective workload of the user during the respective prior shift. In one embodiment, each set of user data (e.g., corresponding to a given user at a given time/during a given shift) may be referred to as an exemplar or a training exemplar.

At block 910, the interface system similarly determines a stress measure for each exemplar of user data. That is, for each set of user data corresponding to a given user during a given shift/at a given time, the interface system can generate, receive, or otherwise determine a stress measure for the user at the given time. In some embodiments, the stress measure is user provided. For example, the user themselves may indicate (during or after the shift or time) the level of stress they are or were feeling. As discussed above, this stress measure can be used as a target output for the stress model.

At block 915, the interface system updates the stress model based on each exemplar in the user data. For example, as discussed above, the interface system may process the user data as input to generate a predicted stress score, and compare this stress score to the stress measure determined at block 910. Based on the difference between these scores, the interface system can refine the parameters of the stress model (e.g., updating the weights of a neural network) to generate more accurate stress scores (e.g., scores that more closely mirror the user-reported stress). In this way, the interface system can train the stress model iteratively.

At block 920, the interface system determines whether there are additional user data (e.g., additional exemplars) remaining that have not-yet been used to refine the model. If so, the method 900 returns to block 905. If not, the method 900 continues to block 925, where the interface system deploys the model (e.g., locally or to one or more other systems) for inferencing. That is, after training, the interface system (or another system) can use the stress model to generate predicted stress levels during runtime, allowing the interface system (or other systems) to continuously update dynamic interfaces to respond to the user's stress.

Although depicted as a sequential and iterative process for conceptual clarity (e.g., where each exemplar is used to refine the model in turn, such as using stochastic gradient descent), in some aspects, the interface system may use one or more exemplars in a batch to refine the model simultaneously (e.g., using batch gradient descent). Additionally, though the illustrated example depicts collection/generation of the training data while training the model, in some aspects, the exemplars may be generated and/or labeled in a batch, and then used to refine the model once all exemplars are ready.

### Example Method for Refining Stress Models Based on Interface Feedback

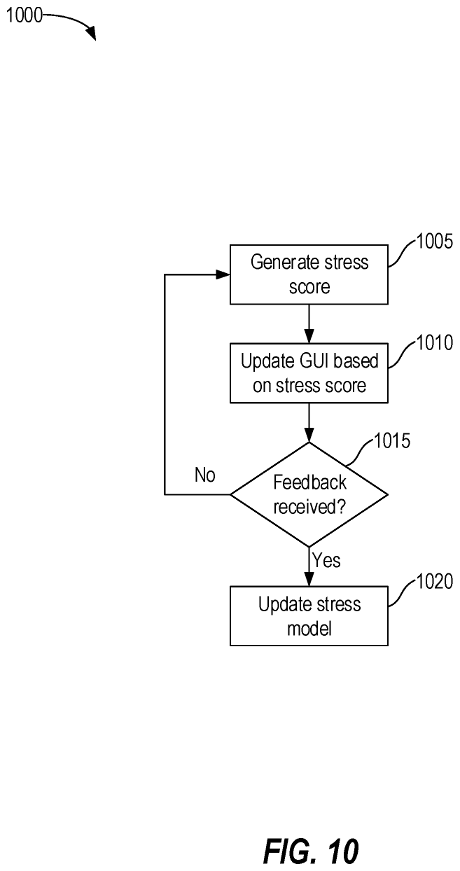

FIG. 10 is a flow diagram depicting an example method 1000 for refining stress models based on interface feedback. In some embodiments, the method 1000 is performed by an interface system, such as the interface system 115 of FIG. 1, the interface system 230 of FIG. 2, and/or the interface system 315 of FIG. 3. In some embodiments, the method 1000 is performed by a user device (or by an interface system integrated into a user device).

At block 1005, the interface system generates a stress score for the user. For example, as discussed above with reference to blocks 805, 810, and 815 of FIG. 8, the interface system can collect user data for the user that is currently interacting with the interface, and process this data using a stress model (e.g., a trained machine learning model) to generate a stress score for the user. In some aspects, as discussed above, the user data may include workload information (e.g., information relating to the current shift the user is working), as well as other information such as how quickly they are dismissing prompts on the interface, how much pressure they are applying to buttons or a touchscreen of the user device, their facial expression and/or tone of voice, and the like.

At block 1010, the interface system updates the GUI based on the stress score. For example, as discussed above with reference to block 820 of FIG. 8, the interface system may update the GUI by adding or removing prompts, requesting (or refraining from requesting) further interaction or confirmation, and the like. As non-limiting examples, the interface system may request action such as selecting a specified visual object or element on the GUI, typing or speaking a specific word or phrase, scrolling down or moving to a different part of the interface, and the like. In some embodiments, as discussed above, the interface system may update the GUI to output information relating to at-risk patients, and/or to suggest or instruct the user to wait, meditate, breathe, or take other actions before proceeding.

At block 1015, the interface system determines whether user feedback has been received. This may include, for example, feedback on the updated GUI (e.g., indicating whether it is appropriate or helpful), feedback indicating the user's current stress level, feedback indicating how quickly the user dismissed the prompt or otherwise moved past the updated GUI, feedback indicating how much pressure the user is applying to the interface (e.g., to buttons or to a touchscreen), feedback indicating the user's facial expression and/or voice characteristics (e.g., tone, speed, volume, etc.), and the like.

If no feedback has been received, the method 1000 returns to block 1005 to generate a revised stress score for the user (or a new stress score for a new user using the interface). In this way, the interface system can loop and continuously update the GUI in a dynamic manner based on user stress levels.

If feedback was received, the method 1000 continues to block 1020, where the interface system updates the stress model and/or the GUI generation process. For example, as discussed above, the interface system may use the feedback (e.g., the user-indicated stress) as target output for the model when processing the current user data as input. This can allow the interface system to use the feedback to define a new training exemplar, which can in turn be used to provide continuous learning or refinement of the stress model (either for the specific user, or to refine a global model).

In some aspects, as discussed above, the interface system can additionally or alternatively use the feedback to refine the GUI-generation process, such as to modify or refine one or more thresholds or rules used to determine which interface modification(s) should be used (e.g., whether to request that the user click a button or type a string), as well as the magnitude of these modifications (e.g., whether to enforce a five-second pause before continuing or a ten-second pause, or whether to request that the user type a 3-letter phrase or a 7-letter phrase).

In this way, the interface system can dynamically update its models in order to ensure that it can continue to provide accurate and reliable stress scores and useful and efficient interfaces, thereby improving the operations of the system.

### Example Method for Processing Data with a Stress Model

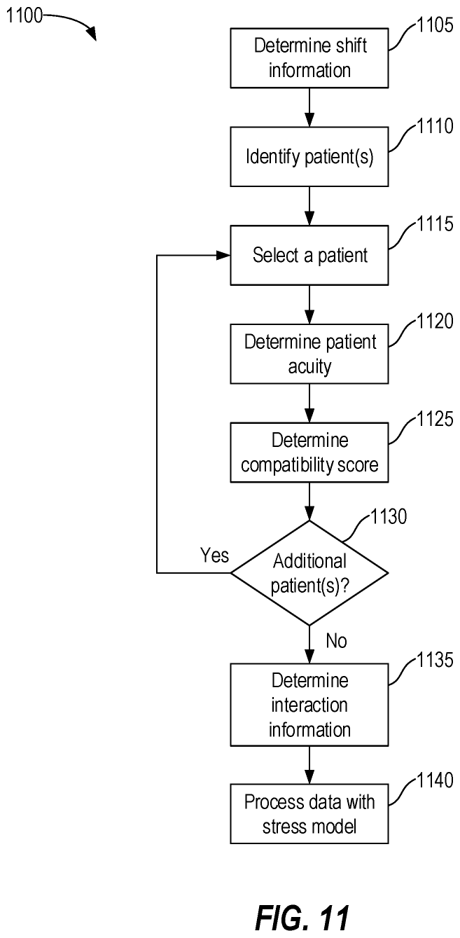

FIG. 11 is a flow diagram depicting an example method for processing data with a stress model. In some embodiments, the method 1100 is performed by an interface system, such as the interface system 115 of FIG. 1, the interface system 230 of FIG. 2, and/or the interface system 315 of FIG. 3. In some embodiments, the method 1100 is performed by a user device (such as the user device 110 of FIG. 1 and/or the user device 325 of FIG. 3), or by an interface system integrated into a user device. In some embodiments, the method 1100 provides additional detail for blocks 810 and 815 of FIG. 8.

At block 1105, the interface system determines current shift information for the user that is currently interacting with the interface. For example, as discussed above, the interface system may determine the length of the current shift, how much time has elapsed since the start of the shift, how much time remains until the end of the shift, what the current time is, whether the shift is a first shift, second shift, or third shift, and the like. In some embodiments, the interface system can similarly determine what tasks the user has performed (or will perform) during the current shift, such as the number of times they have helped a patient use the toilet, the number of times they have changed a patient's bedding or bandaging, and the like. In some embodiments, the interface system can further determine information about the patients, such as the number of patients the user is caring for, has cared for, or will care for during the current shift.

At block 1110, the interface system identifies the set of patient(s) that the user is caring for, has cared for, or will care for during the current shift. At block 1115, the interface system then selects one of these patients for evaluation. Generally, this selection may be performed using any suitable criteria or operation (including randomly or pseudo-randomly), as the interface system will evaluate each patient in turn. Further, though depicted as a sequential process for conceptual clarity, in some aspects, the interface system can select and evaluate multiple patients in parallel.

At block 1120, the interface system determines the acuity of the selected patient. For example, as discussed above, the interface system (or another system) may process one or more patient attributes (e.g., their conditions, diagnoses, demographics, weight, age, and the like) using an acuity model (e.g., a trained machine learning model and/or a static rules-based model) to generate an acuity score that quantifies the acuity of the patient (e.g., the amount of care they require). In one embodiment, the acuity score is positively correlated with the patient's needs, such that higher scores indicate that the patient requires more intensive assistance or care (and therefore more work from the user).

At block 1125, the interface system can optionally determine a compatibility score for the patient, with respect to the user. For example, the interface system (or another system) may process one or more patient attributes (e.g., their conditions, diagnoses, demographics, weight, age, personality information, and the like) and one or more user attributes of the user (e.g., their preferences, demographics, personality information, and the like) using a compatibility model (e.g., a trained machine learning model and/or a static rules-based model) to generate a compatibility score that quantifies how compatible the patient and user are (e.g., the amount of discord experienced when they interact). In one embodiment, the compatibility score is negatively correlated with the patient's needs, such that higher scores indicate that the patient will generally cause less stress and work for the user.

At block 1130, the interface system determines whether one or more additional patients remain to be evaluated. If so, the method 1100 returns to block 1115. If all of the patients that the user is or will be assisting during the current shift have been evaluated, the method 1100 continues to block 1135.

At block 1135, the interface system determines interaction information for the user. As discussed above, the interaction information can generally provide detail relating to the user's current interaction with the interface. For example, the interface system may determine their facial expression, tone of voice, volume and/or speed of speech, how quickly they are dismissing prompts and/or moving through the interface, the pressure they are using on a touchscreen or button, and the like. This information may be informative of the user's current stress level and emotional and mental state.

At block 1140, the interface system can process all of the determined user data (e.g., the shift information determined in block 1105, the patient-specific information determined in blocks 1120 and 1125, and/or the interaction information determined in block 1135) using the stress model in order to generate a current stress score. As discussed above, this stress score can generally indicate the predicted stress level of the user, and/or the probability that the user's current stress level meets or exceeds a defined level. This information can, in turn, be used to refine or update the interface in order to ensure that the process proceeds smoothly.

### Example Method for Modifying Interfaces Based on Patient Information and User Context

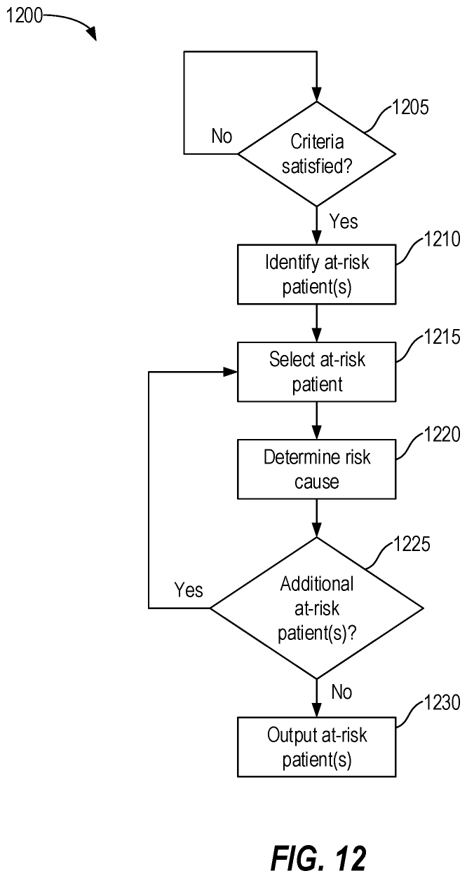

FIG. 12 is a flow diagram depicting an example method for modifying interfaces based on patient information and user context. In some embodiments, the method 1200 is performed by an interface system, such as the interface system 115 of FIG. 1, the interface system 230 of FIG. 2, and/or the interface system 315 of FIG. 3. In some embodiments, the method 1100 is performed by a user device (such as the user device 110 of FIG. 1 and/or the user device 325 of FIG. 3), or by an interface system integrated into a user device. In one embodiment, the method 1200 provides additional detail for block 820 of FIG. 8.

At block 1205, the interface system determines whether one or more defined criteria are satisfied. For example, the interface system may determine whether the current user stress score meets or exceeds a defined threshold. In some aspects, the interface system determines whether the stress level (or other information) indicates that the user is at particular risk of acting negligently or in some way that may cause harm to a patient, such as because the predicted stress level is very high (e.g., above the highest threshold).

At block 1210, the interface system identifies any at-risk patients being cared for by the user. For example, the interface system may evaluate the patient data for patients that the user is caring for or will care for during their shift, and determine whether any of these patients can be classified as “at-risk” or “vulnerable.” In some embodiments, as discussed above, the interface system can identify the at-risk patients by determining whether each has one or more conditions, diagnoses, or other attributes or characteristics from a defined list of attributes classified as “at-risk” attributes. In some embodiments, the interface system can determine the at-risk patients using one or more trained models, such as the acuity model discussed above. Generally, the interface system may define patients as “at-risk” or vulnerable if, due to their particular situation (e.g., mental state), they are particularly susceptible or influenced by the user's stress or actions.

At block 1215, the interface system selects one of the at-risk patients (if any were identified). Generally, this selection can be performed using any suitable technique or operation (including random or pseudo-random selection), as each will be processed in turn. Additionally, though depicted as a sequential process for conceptual clarity, in some aspects, the interface system can evaluate some or all of the at-risk patients in parallel.

At block 1220, the interface system determines the condition or attribute, of the selected patient, that causes them to be considered at-risk or vulnerable. For example, as discussed above, the interface system can identify which attribute(s), from the list of vulnerable attributes, the patient possesses.

At block 1225, the interface system can determine whether there is at least one additional at-risk patient that has not-yet been evaluated. If so, the method 1200 returns to block 1215. If not, the method 1200 continues to block 1230.

At block 1230, the interface system updates the GUI to output an indication of the at-risk patient(s) and/or the determined risk cause(s). For example, as discussed above, the interface may include a prompt or warning that the current or future patients, with whom the user will interact, are at particular risk due to the indicated condition(s), and suggest or instruct the user to perform one or more actions prior to interacting with them (such as taking a deep breath, meditating, taking a break, finding another user or caregiver to substitute for them, and the like).

In this way, the interface system can significantly reduce harm to patients based on the generated stress scores. This improves not only the patient outcomes, but also the operations of the computing system (e.g., by only performing the method 1200 when the stress score satisfies the criteria, thereby reducing computational expense when it does not).

### Example Method for Modifying User Interfaces

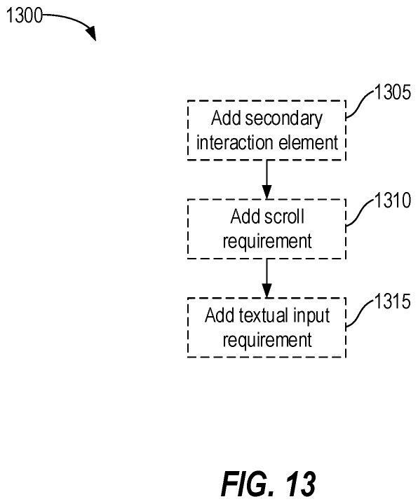

FIG. 13 is a flow diagram depicting an example method 1300 for modifying user interfaces. In some embodiments, the method 1300 is performed by an interface system, such as the interface system 115 of FIG. 1, the interface system 230 of FIG. 2, and/or the interface system 315 of FIG. 3. In some embodiments, the method 1300 is performed by a user device (such as the user device 110 of FIG. 1 and/or the user device 325 of FIG. 3), or by an interface system integrated into a user device. In some embodiments, the method 1300 is performed by a user device (or by an interface system integrated into a user device). In one embodiment, the method 1300 provides additional detail for block 820 of FIG. 8.

At block 1305, the interface system can optionally add a secondary interaction element to the interface, as compared to a default interface. For example, as discussed above with reference to FIG. 4, the interface system may add a prompt requesting that the user select a specified visual element, such as a shape or a picture of the patient, prior to proceeding. Similarly, the interface system may add a secondary confirmation (e.g., another prompt after the first one is dismissed).

At block 1310, the interface system can optionally add a scroll or other movement requirement, as compared to a default interface. For example, as discussed with reference to FIG. 5, the interface system may add a scroll bar and instruct the user to scroll to the bottom of the page before proceeding. This additional action can cause the user to slow down and generally improve their accuracy.

At block 1315, the interface system can optionally add a textual input requirement, as compared to the default interface. For example, as discussed above with reference to FIG. 6, the interface system may instruct the user to type in a specific word or phrase in order to proceed.

The indicated requests in the depicted method 1300 are a few examples of interface modifications that can be performed based on detected stress levels. As discussed above, however, there may be any number and variety of interface modifications and prompts that can be used in various aspects. Additionally, in some aspects, two or more prompts may be combined (e.g., requiring that the user scroll to the bottom of the prompt, wait five seconds, and then manually type in “Ok” before proceeding). Using the method 1300, the interface system can provide significantly improved interfaces and reduce errors.

### Example Method for Generating Dynamic Interfaces

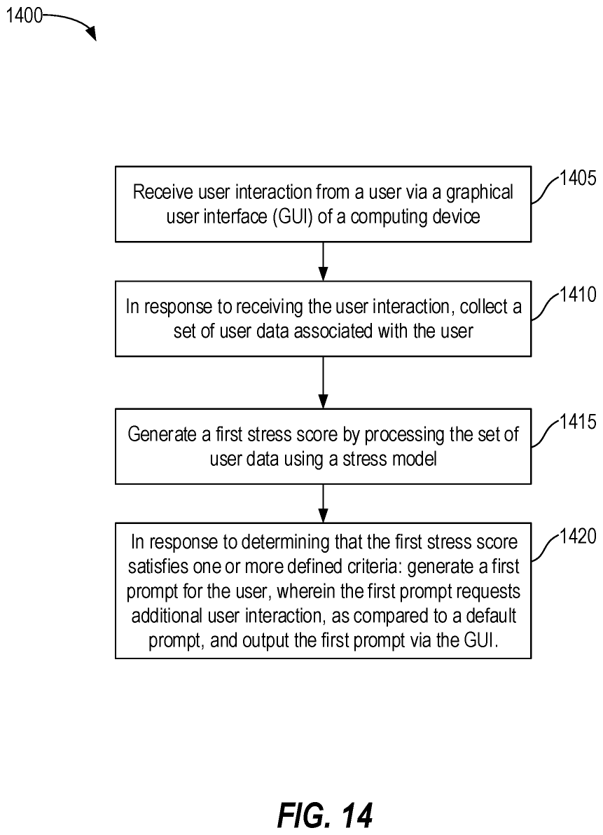

FIG. 14 is a flow diagram depicting an example method 1400 for outputting dynamic prompts via user interfaces. In some embodiments, the method 1400 is performed by an interface system, such as the interface system 115 of FIG. 1, the interface system 230 of FIG. 2, and/or the interface system 315 of FIG. 3. In some embodiments, the method 1400 is performed by a user device (such as the user device 110 of FIG. 1 and/or the user device 325 of FIG. 3), or by an interface system integrated into a user device.

At block 1405, user interaction is received from a user via a graphical user interface (GUI) of a computing device (e.g., user device 110 of FIG. 1 and/or user device 325 of FIG. 3).

At block 1410, in response to receiving the user interaction, a set of user data (e.g., user data 120 of FIG. 1 and/or user data 305 of FIG. 3) associated with the user is collected.

At block 1415, a first stress score is generated by processing the set of user data using a stress model (e.g., stress model 235 of FIG. 2).

At block 1420, in response to determining that the first stress score satisfies one or more defined criteria: a first prompt for the user is generated (e.g., updated GUI 320 of FIG. 3), wherein the first prompt requests additional user interaction, as compared to a default prompt, and the first prompt is output via the GUI.

### Example User System for Improved User Interfaces

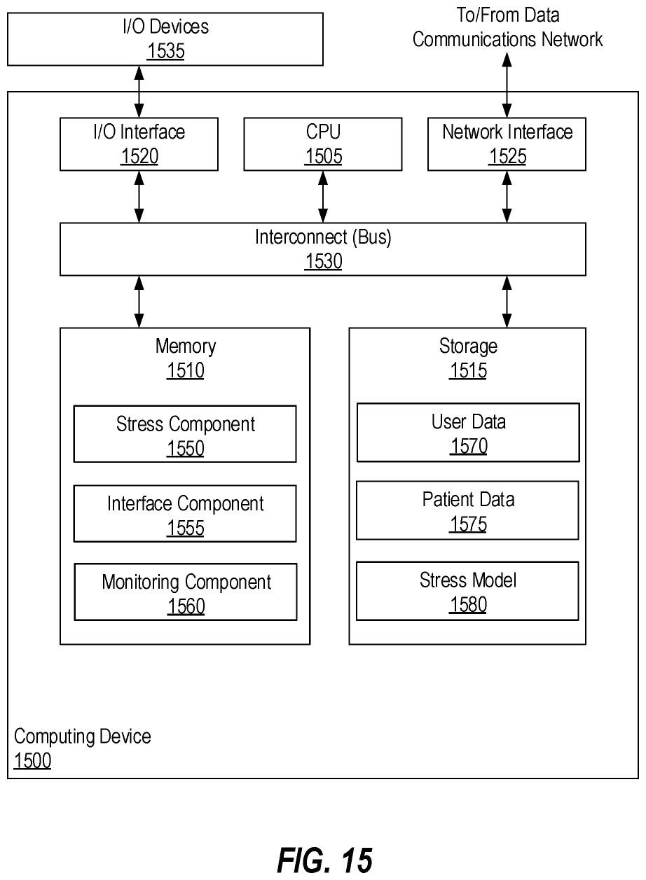

FIG. 15 depicts an example computing device 1500 configured to perform various aspects of the present disclosure. Although depicted as a physical device, in embodiments, the computing device 1500 may be implemented using virtual device(s), and/or across a number of devices (e.g., in a cloud environment). In one embodiment, the computing device 1500 corresponds to one or more systems in a healthcare platform, such as an interface system (e.g., interface system 115 of FIG. 1, the interface system 230 of FIG. 2, and/or the interface system 315 of FIG. 3) and/or a user device (such as the user device 110 of FIG. 1 and/or the user device 325 of FIG. 3).

As illustrated, the computing device 1500 includes a CPU 1505, memory 1510, storage 1515, a network interface 1525, and one or more I/O interfaces 1520. In the illustrated embodiment, the CPU 1505 retrieves and executes programming instructions stored in memory 1510, as well as stores and retrieves application data residing in storage 1515. The CPU 1505 is generally representative of a single CPU and/or GPU, multiple CPUs and/or GPUs, a single CPU and/or GPU having multiple processing cores, and the like. The memory 1510 is generally included to be representative of a random access memory. Storage 1515 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1535 (such as keyboards, monitors, etc.) are connected via the I/O interface(s) 1520. Further, via the network interface 1525, the computing device 1500 can be communicatively coupled with one or more other devices and components (e.g., via a network, which may include the Internet, local network(s), and the like). As illustrated, the CPU 1505, memory 1510, storage 1515, network interface(s) 1525, and I/O interface(s) 1520 are communicatively coupled by one or more buses 1530.

In the illustrated embodiment, the memory 1510 includes a stress component 1550, an interface component 1555, and a monitoring component 1560, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1510, in embodiments, the operations of the depicted components (and others not illustrated) may be implemented using hardware, software, or a combination of hardware and software.

In one embodiment, the stress component 1550 may be used to predict user stress levels (e.g., by processing user data such as workload information using machine learning), as discussed above. For example, the stress component 1550 may be used to train stress models, and/or to collect user data (e.g., workload data) to generate predicted stress using a trained stress model. The interface component 1555 may generally be used to determine which interface modification(s) should be used (e.g., based on the generated stress level), and/or to generate or modify the interface accordingly (e.g., to include additional interaction requests), as discussed above. For example, the interface component 1555 may determine that the stress score meets or exceeds a given threshold, and generate an updated interface including further information or requests based on this determination. The monitoring component 1560 may generally be used to monitor user interactions with the interface to identify or generate feedback, such as based on applied pressure, speech of the user, facial expressions of the user, user-indicated stress, and the like. As discussed above, this feedback can be used to train or refine the models.

In the illustrated example, the storage 1515 includes user data 1570, patient data 1575, and one or more stress models 1580. In one embodiment, the user data 1570 may include attributes or characteristics of the users, such as their current, prior, and/or future workloads, their preferences, and the like. The patient data 1575 can generally include attributes of the patient(s) being cared for, such as their conditions, diagnoses, demographics, acuity score, and the like. The stress model 1580 generally corresponds to a computational model (e.g., a machine learning model) that generates a predicted stress score or measure based on input data. Although depicted as residing in storage 1515, the user data 1570, patient data, and stress model 1580 may be stored in any suitable location, including memory 1510.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., interface system 115 of FIG. 1, interface system 230 of FIG. 2, and/or interface system 315 of FIG. 3) or related data available in the cloud. For example, the interface system could execute on a computing system in the cloud and automatically generate stress predictions sand dynamic interfaces based on user data. In such a case, the interface system could use context-specific user data to generate current stress predictions, and return customized and dynamic interfaces that improve operations. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

### Example Clauses

Implementation examples are described in the following numbered clauses:

Clause 1: A method, comprising: receiving user interaction from a user via a graphical user interface (GUI) of a computing device; in response to receiving the user interaction, collecting a set of user data associated with the user; generating a first stress score by processing the set of user data using a stress model; and in response to determining that the first stress score satisfies one or more defined criteria: generating a first prompt for the user, wherein the first prompt requests additional user interaction, as compared to a default prompt; and outputting the first prompt via the GUI.

Clause 2: The method of Clause 1, wherein the set of user data comprises workload information for a current job shift that the user is currently working.

Clause 3: The method of any one of Clauses 1-2, wherein the workload information comprises one or more of: (i) a duration of the current job shift, (ii) an amount of time that has elapsed during the current job shift, (iii) an amount of time that remains during the current job shift, or (iv) a current time.

Clause 4: The method of any one of Clauses 1-3, wherein: the user is a healthcare worker, and the workload information comprises a set of acuity scores for a set of patients being cared for by the user during the current job shift.

Clause 5: The method of any one of Clauses 1-4, wherein the set of acuity scores is generated by, for each patient in the set of patients, processing corresponding patient data using a machine learning model trained to predict patient acuity.

Clause 6: The method of any one of Clauses 1-5, wherein generating the first prompt comprises: identifying one or more at-risk patients, from the set of patients, based on patient data; and indicating the one or more at-risk patients in the first prompt.

Clause 7: The method of any one of Clauses 1-6, wherein the stress model is a trained machine learning model, the method further comprising training the stress model, comprising: collecting a training set of user data associated with a historic user; determining a level of stress being experienced by the historic user; generating a test stress score by processing the training set of user data using the stress model; and refining the stress model based on a difference between the test stress score and the determined level of stress.

Clause 8: The method of any one of Clauses 1-8, wherein the additional user interaction comprises at least one of: (i) selecting a specified visual element of the first prompt prior to dismissing the first prompt, (ii) scrolling from a first portion of the first prompt to a second portion of the first prompt prior to dismissing the first prompt, or (iii) typing a specified textual string prior to dismissing the first prompt.

Clause 10: A system, comprising: a memory comprising computer-executable instructions; and one or more processors configured to execute the computer-executable instructions and cause the processing system to perform a method in accordance with any one of Clauses 1-9.

Clause 11: A system, comprising means for performing a method in accordance with any one of Clauses 1-9.

Clause 12: A non-transitory computer-readable medium comprising computer-executable instructions that, when executed by one or more processors of a processing system, cause the processing system to perform a method in accordance with any one of Clauses 1-9.

Clause 13: A computer program product embodied on a computer-readable storage medium comprising code for performing a method in accordance with any one of Clauses 1-9.

What is claimed is:

1. A method, comprising:
receiving user interaction from a user via a graphical user interface (GUI) of a computing device;
in response to receiving the user interaction, collecting a set of user data associated with the user;
generating a first stress score by processing the set of user data using a stress model; and
in response to determining that the first stress score satisfies one or more defined criteria:
generating a first prompt for the user, wherein the first prompt requests additional user interaction, as compared to a default prompt; and
outputting the first prompt via the GUI.

2. The method of claim 1, wherein the set of user data comprises workload information for a current job shift that the user is currently working.

3. The method of claim 2, wherein the workload information comprises one or more of:
(i) a duration of the current job shift,
(ii) an amount of time that has elapsed during the current job shift,
(iii) an amount of time that remains during the current job shift, or
(iv) a current time.

4. The method of claim 2, wherein:
the user is a healthcare worker, and
the workload information comprises a set of acuity scores for a set of patients being cared for by the user during the current job shift.

5. The method of claim 4, wherein the set of acuity scores is generated by, for each patient in the set of patients, processing corresponding patient data using a machine learning model trained to predict patient acuity.

6. The method of claim 4, wherein generating the first prompt comprises:
identifying one or more at-risk patients, from the set of patients, based on patient data; and
indicating the one or more at-risk patients in the first prompt.

7. The method of claim 1, wherein the stress model is a trained machine learning model, the method further comprising training the stress model, comprising:
collecting a training set of user data associated with a historic user;
determining a level of stress being experienced by the historic user;
generating a test stress score by processing the training set of user data using the stress model; and
refining the stress model based on a difference between the test stress score and the determined level of stress.

8. The method of claim 1, wherein the additional user interaction comprises at least one of:
(i) selecting a specified visual element of the first prompt prior to dismissing the first prompt,
(ii) scrolling from a first portion of the first prompt to a second portion of the first prompt prior to dismissing the first prompt, or
(iii) typing a specified textual string prior to dismissing the first prompt.

9. A non-transitory computer-readable medium comprising computer-executable instructions that, when executed by one or more processors of a processing system, cause the processing system to perform an operation comprising:
receiving user interaction from a user via a graphical user interface (GUI) of a computing device;
in response to receiving the user interaction, collecting a set of user data associated with the user;
generating a first stress score by processing the set of user data using a stress model; and
in response to determining that the first stress score satisfies one or more defined criteria:
generating a first prompt for the user, wherein the first prompt requests additional user interaction, as compared to a default prompt; and
outputting the first prompt via the GUI.

10. The non-transitory computer-readable medium of claim 9, wherein the set of user data comprises workload information for a current job shift that the user is currently working, and wherein the workload information comprises one or more of:
(i) a duration of the current job shift,
(ii) an amount of time that has elapsed during the current job shift,
(iii) an amount of time that remains during the current job shift, or
(iv) a current time.

11. The non-transitory computer-readable medium of claim 10, wherein:
the user is a healthcare worker,
the workload information comprises a set of acuity scores for a set of patients being cared for by the user during the current job shift, and
the set of acuity scores is generated by, for each patient in the set of patients, processing corresponding patient data using a machine learning model trained to predict patient acuity.

12. The non-transitory computer-readable medium of claim 11, wherein generating the first prompt comprises:
identifying one or more at-risk patients, from the set of patients, based on patient data; and
indicating the one or more at-risk patients in the first prompt.

13. The non-transitory computer-readable medium of claim 9, wherein the stress model is a trained machine learning model, the operation further comprising training the stress model, comprising:
collecting a training set of user data associated with a historic user;
determining a level of stress being experienced by the historic user;
generating a test stress score by processing the training set of user data using the stress model; and
refining the stress model based on a difference between the test stress score and the determined level of stress.

14. The non-transitory computer-readable medium of claim 9, wherein the additional user interaction comprises at least one of:
(i) selecting a specified visual element of the first prompt prior to dismissing the first prompt,
(ii) scrolling from a first portion of the first prompt to a second portion of the first prompt prior to dismissing the first prompt, or
(iii) typing a specified textual string prior to dismissing the first prompt.

15. A system, comprising:
a memory comprising computer-executable instructions; and
one or more processors configured to execute the computer-executable instructions and cause the system to perform an operation comprising:
receiving user interaction from a user via a graphical user interface (GUI) of a computing device;
in response to receiving the user interaction, collecting a set of user data associated with the user;
generating a first stress score by processing the set of user data using a stress model; and
in response to determining that the first stress score satisfies one or more defined criteria:
generating a first prompt for the user, wherein the first prompt requests additional user interaction, as compared to a default prompt; and
outputting the first prompt via the GUI.

16. The system of claim 15, wherein the set of user data comprises workload information for a current job shift that the user is currently working, and wherein the workload information comprises one or more of:
(i) a duration of the current job shift,
(ii) an amount of time that has elapsed during the current job shift,
(iii) an amount of time that remains during the current job shift, or
(iv) a current time.

17. The system of claim 16, wherein:
the user is a healthcare worker,
the workload information comprises a set of acuity scores for a set of patients being cared for by the user during the current job shift, and
the set of acuity scores is generated by, for each patient in the set of patients, processing corresponding patient data using a machine learning model trained to predict patient acuity.

18. The system of claim 17, wherein generating the first prompt comprises:
identifying one or more at-risk patients, from the set of patients, based on patient data; and
indicating the one or more at-risk patients in the first prompt.

19. The system of claim 15, wherein the stress model is a trained machine learning model, the operation further comprising training the stress model, comprising:
collecting a training set of user data associated with a historic user;
determining a level of stress being experienced by the historic user;
generating a test stress score by processing the training set of user data using the stress model; and
refining the stress model based on a difference between the test stress score and the determined level of stress.

20. The system of claim 15, wherein the additional user interaction comprises at least one of:
(i) selecting a specified visual element of the first prompt prior to dismissing the first prompt,
(ii) scrolling from a first portion of the first prompt to a second portion of the first prompt prior to dismissing the first prompt, or
(iii) typing a specified textual string prior to dismissing the first prompt.
